(19) United States
(12) Patent Application Publication
(10) Pub. No.: US 2024/0415775 A1
(43) Pub. Date: Dec. 19, 2024

(54) Multi-Layered Dextrose Tablets

(71) Applicant: Fertin Pharma A/S, Vejie (DK)

(72) Inventor: Helle Wittorff, Vejle Ost (DK)

(73) Assignee: Fertin Pharma A/S, Vejle (DK)

(21) Appl. No.: 18/686,557

(22) PCT Filed: Oct. 12, 2022
(86) PCT No.: PCT/DK2022/050215

§ 371 Date: Aug. 20, 2024

(51) Int. Cl. A61K 9/24; A61K 9/20; A61K 31/137; A61K 31/167; A61K 31/198; A61K 31/375; A61K 31/485; A61K 31/522; A61K 36/258

(52) U.S. Cl. CPC A61K 9/209; A61K 9/2018; A61K 9/2054; A61K 31/137; A61K 31/167; A61K 31/198; A61K 31/375; A61K 31/485; A61K 31/522; A61K 36/258

(57) ABSTRACT

An oral chewable tablet contains dextrose in an amount from 50 to 95% by weight of the tablet; and one or more active ingredients in an amount from 0.1 to 50% by weight of the tablet. The oral chewable tablet includes at least two compressed layers containing the dextrose and the one or more active ingredients, the first of the compressed layers being cohered to and adjacent to the second layer.

### FIELD OF THE INVENTION

The invention relates to the field of chewable tablets for oral delivery of active ingredients. In particular, the invention relates to multi-layered tablets suitable for high load delivery of active ingredients.

### BACKGROUND OF THE INVENTION

Various attempts have been proposed in the past for incorporating a high load of active ingredients in oral tablets. While these solutions may provide some benefits with respect to containing a high content of actives in the tablets, clear drawbacks are frequently seen that prevent the solutions from being marketed.

Particularly relevant for tablets with a high load of active ingredients, challenges often arise during the manufacturing process. Often, the high load of active ingredients provides fragile tablets that may cause processing problems in the manufacturing process, during storage and transportation of the tablets.

Especially, multi-layered tablets may be difficult to produce with suitable results. Often, these tablets are friable and therefore less preferred in terms of disadvantages of storing and transporting the tablets. Typically, when multi-layered tablets are prepared in a tableting machine, the compression process gives some limitations on the amount of actives to be incorporated in the layers of the tablets,

For instance, a pre-compression step in the tableting procedure for a first layer of the tablets may give rise to problems with friability once a second layer is applied on top of the first layer. Usually, it is necessary with a high pressure for a second layer in order to provide a suitable tablet that forms a cohered and enclosed structure of a multi-layered tablet, which is robust.

Also, a high load of active ingredients may cause issues for the overall sensorial properties of multi-layered tablets. Both the nature of the active ingredients with respect to taste properties and pronounced bitterness for some active ingredients, and the high amount of actives relative to other ingredients in the multi-layered tablets, may be a challenge to formulation specialists.

One of the more critical issues is that it is often difficult to formulate multi-layered tablets for oral delivery with suitable sensorial properties. Sensorial properties in the present context include the overall mouthfeel of the multi-layered tablet when chewed and the resulting delivery of the active ingredients in the oral cavity. For instance, a pronounced powdery sense is often not convenient to the user and as a result may often provide inferior multi-layered tablets that are not used on a frequent basis.

Preferably, a multi-layered tablet is to be provided that may also help in obtaining improved sensorics properties. Here, important sensorics properties include friability, texture, flavor perception, sweetness perception and off-notes associated with the actives. These properties are both relevant from a convenience perspective in chewable multi-layered tablets, but certainly also in order to support an appropriate delivery of actives from the multi-layered tablets and avoid adverse side effects of the actives.

One of the challenges with chewable multi-layered tablets as a delivery vehicle of actives is that actives may tend to be associated with off-notes during administration due to the specific physiochemical properties of the compounds. The taste masking challenge is more profound when a higher release of the actives are delivered by such multi-layered tablets. If off-notes are the predominant sensation during administration, convenience may be affected and even more critically, the delivery of the actives may also be affected. Saliva production may be suppressed, and the delivery vehicle may not be handled correctly.

Further, even when sensorial properties to some degree are complied with, there would usually also be a desire for relative fast delivery of active ingredients upon oral administration. Often, this desire of a relative fast release is counteracted by the desire for taste-masking of the active ingredients.

Particularly, less attention is given on the benefits of chewable multi-layered tablet formulations that may help in obtaining a release characteristic of actives that offers increased convenience and effectiveness. One of these release characteristics is increased generation of saliva upon chewing. Increased saliva generation and particularly an experience of increased saliva generation upon administration may have some pronounced benefits for delivery of actives.

Accordingly, there is a need for multi-layered tablets for oral delivery of active ingredients that both accommodate beneficial sensorial properties and at the same time accommodate a relative fast delivery of active ingredients. This may be particularly desired for active ingredients in a high load but may also be desired for active ingredients in a low load, such as ingredients with pronounced bitterness properties.

### SUMMARY OF THE INVENTION

Accordingly, there is provided an oral chewable tablet, comprising:
dextrose in an amount from 50 to 95% by weight of the tablet; and
one or more active ingredients in an amount from 0.1 to 50% by weight of the tablet, wherein the oral chewable tablet comprises:
at least two compressed layers containing said dextrose and the one or more active ingredients, the first of the compressed layers being cohered to and adjacent to the second layer.

Providing a chewable multi-layered tablet according to the invention may solve various problems of the prior art and aims at establishing a chewable multi-layered tablet that combines beneficial delivery properties of actives combined with advantageous sensorial properties.

Generally, the aim of the oral chewable multi-layered tablets according to the invention is the combination of a relatively high amount of dextrose and one or more active ingredients in a configuration that at the same time allows for more flexibility than a conventional oral chewable one-layered tablet.

One advantage of the invention is a surprisingly strong saliva generation compared to conventional chewable tablets and lozenges. Increased generation of saliva may have a huge impact on the delivery of the one or more active ingredients. Specifically, when increased generation of saliva is coordinated with release of the one or more active ingredients from the multi-layered tablet, relatively quick delivery is obtained. Hence, a synergy between the effect of the one or more active ingredients and increased saliva generation may be seen according to the invention.

One unexpected advantage over the prior art is that the saliva generation is surprisingly sustained even after a user has swallowed the bulk-portion of the dextrose. This sustaining of the salivation generation may be advantageous in relation to many applications of a chewable multi-layered tablet ranging from mouthfeel, taste, flavor perception, etc.

With respect to release properties, the present invention may offer an improved release profile of the one or more active ingredients compared to conventional chewable one-layered tablets. In particular, the specific tablet formulation platform of the present invention may serve to provide improved release characteristics of the one or more active ingredients compared to conventional chewable one-layered tablet formulation platforms applied in combination with one or more active ingredients.

A very important aspect of the present invention is the provision of beneficial sensorial properties. Here, important sensorial properties include mouthfeel, ease of chewing/melting into liquid, friability (mechanical robustness), texture, flavor perception, sweetness perception and off-notes associated with the one or more active ingredients. These properties are both relevant from a convenience perspective in chewable multi-layered tablets, but certainly also in order to support an appropriate delivery of the one or more active ingredients from the chewable multi-layered tablet formulation, such as an improved release profile, and to avoid adverse side effects of the one or more active ingredients.

The present inventor has shown very surprising results with the specific combination of features of the present invention in terms of these sensorial properties. It was an unexpected result that the invention could both contribute to an improved release profile, such as rapid release of the one or more active ingredients, and at the same time provide very beneficial sensorics properties which in terms may also support an appropriate delivery of the one or more active ingredients from the chewable multi-layered tablet.

One of the sensorial properties that is particularly advantageous is the mouthfeel. The mouthfeel of the oral multi-layered tablet during use is critical for the release of the one or more active ingredients and the experience as well as convenience during use. The mouthfeel was improved which was not expected by the inventor of the present invention.

Also, it was not expected that a suitable friability and other properties could be obtained by combining dextrose in a relative high amount according to the invention with the one or more active ingredients of the invention in a multi-layered configuration. It is critical that friability is balanced. Without being bound by theory it was expected that the layers of the multi-layered tablet would not adhere properly or at least cause breaking during the compression step as well as capping or de-lamination upon ejecting the tablets from the tablet press. However, surprising results were obtained for the multi-layered tablets according to the invention with respect to friability as well as sensorial properties.

In context of the present invention, a “chewable tablet” is intended to mean an oral tablet that is chewed upon oral administration, having characteristics allowing convenient chewing without adverse side effects associated with the texture of the oral tablet.

Providing a layered tablet structure according to the invention may solve various problems of the prior art and aims at establishing a platform that combines beneficial delivery properties of actives combined with advantageous sensorial properties.

In the present context, a ‘layer’ is to be understood as a matrix resulting from pressing one portion of particles according to the invention. This portion may comprise one or more populations of particles. Hence, when a portion of particles according to the invention is applied in the tableting apparatus, and this portion is pressed into a coherent tablet, this would correspond to one ‘layer’ or a ‘first layer’. Optionally, such layer may be pressed in two steps with varying pressure.

Typically, a pre-compression pressure is applied to the first portion being pressed. This pressure will typically be lower than for a ‘second layer’. Pre-compression serves various purposes, such as aligning the first portion with a sharp visual line in the periphery of the multi-layered tablet. Another purpose of substantial character is that it serves to provide a solid support for a “second layer”, thereby establishing bonds between the particles of the material.

Once another portion of particles according to the invention is applied and pressed in the tableting apparatus on top of the already pressed layer, this would correspond to another ‘layer’ or a ‘second layer’. This portion may comprise one or more populations of particles. In the context of the invention, the second layer may also be applied in the tableting apparatus first and the first layer may be applied in the tableting apparatus in a second step.

Due to the inherent nature of conventional tablet pressing, the individual layers of the tableted composition would have a sharp line between the layers when the layers are pressed in two or more subsequent steps. This is seen from a side view of the tablet as distinct layers on top of each other. In an alternative embodiment, two or more portions are applied in subsequent steps to the tableting apparatus and pressed in one sequence. In this case there will not be a sharp line between the layers from a side view, but this would still be considered a layered tablet according to the invention, although the line between the layers would be irregular. Accordingly, in some embodiments, it is not required that layers are processed in separate tableting steps, preferably with a pre-compression step.

In the present context ‘cohered to and adjacent to’ is intended to mean that two layers are pressed together on one top side and one bottom side of two portions of particles comprising one or more populations of particles. Hence, one surface of a layer is attached to one surface of another layer, whereas additional surfaces of the portions are not exposed to each other. Seen from a side view, the layers have a tablet-slice appearance. In some embodiments of the invention, the tablet consists of two layers.

In some embodiments of the invention, the weight ratio of the first layer relative to the second layer is from 1:10 to 10:1.

By adjusting the ‘thicknesses’ of the layers, i.e., the weight ratios, controlled release of the one or more active ingredients may be facilitated. Also, sensorial properties of the tablet may be improved.

In some embodiments of the invention, the weight ratio of the first layer relative to the second layer is from 1:5 to 5:1. In some embodiments of the invention, the weight ratio of the first layer relative to the second layer is from 3:10 to 10:3. In some embodiments of the invention, the weight ratio of the first layer relative to the second layer is from 1:2 to 2:1.

In some embodiments of the invention, tablet consists of three layers where the first layer is the middle layer, and the second layer is cohered to and adjacent to one side of the first layer, and a third layer is cohered to and adjacent to the opposite side of the first layer.

In the present context ‘cohered to and adjacent to’ is intended to mean that two outer layers are located on and attached to one top side and one bottom side of a middle layer. Seen from a side view, the layers have a tablet-slice appearance.

In some embodiments of the invention, the weight ratio of the first layer relative to the second layer relative to the third layer is from 1:5:5 to 10:1:1. In some embodiments of the invention, the weight ratio of the first layer relative to the second layer relative to the third layer is from 1:3:3 to 3:1:1.

By adjusting the ‘thicknesses’ of the layers, i.e., the weight ratios, controlled release of the one or more cannabinoids may be facilitated. Also, sensorial properties of the tablet may be improved.

In some embodiments of the invention, the weight ratio of the first layer relative to the second layer relative to the third layer is from 1:4:4 to 8:1:1. In one embodiment of the invention, the weight ratio of the first layer relative to the second layer relative to the third layer is from 1:4:4 to 6:1:1. In one embodiment of the invention, the weight ratio of the first layer relative to the second layer relative to the third layer is from 1:4:4 to 4:1:1.

According to the invention, tableting implies that the particles are reduced in volume as a result of pressure applied in the tableting apparatus. Hence, while the particles may be free flowing before tableting, once the particles have been pressed, the volume of the particles is reduced, and the particles are cohered together into a continuous matrix, which in the present context is denoted a ‘layer’. In the present context, it is to be understood that the individual particles are not merged after tableting but remain individual ‘discrete’ areas after tableting, constituting the individual particles. Some integration of the particles may be present but generally the particles remains discrete areas of the tablet.

In some embodiments of the invention, the oral chewable tablet is made by direct compression in a tableting machine. In some embodiments of the invention, the first layer is a pre-compressed layer and the second layer is compressed on top of the first layer. In some embodiments of the invention, the first layer is pre-compressed by direct compression in a tableting machine and the second layer is compressed on top of the first layer in the tableting machine. In some embodiments of the invention, the first layer is pre-compressed by direct compression in a tableting machine and the second layer is compressed on top of the first layer in the tableting machine with a higher pressure.

In some embodiments of the invention, the second layer is compressed on top of the first layer with a pressure that is operable to avoid breaking of the tablet during compression. In the present context ‘breaking of the tablet during compression’ is intended to mean the tablet is subject to a fracture during compression in a more or less pronounced degree.

In some embodiments of the invention, the second layer is compressed on top of the first layer with a pressure that is operable to avoid capping of the tablet. Tablet capping is the most common defect. It is when the shaped dome of the tablet fractures away from the body of the tablet. When compressing a tablet, the air is pushed out from between the granules, which allows them to be locked together. If the particles are too dry, over-lubricated or highly elastic, they do not bond together sufficiently. The stress of ejection can open up small microcracks within the compact, which expand and lead to the cap being sheared apart from the body.

In some embodiments of the invention, the second layer is compressed on top of the first layer with a pressure that is operable to avoid de-lamination of the tablet. Lamination of tablets may occur when the product separates into horizontal layers. Lamination is very similar to capping, but occurs in the main body of the tablet, not at the top, and it can occur immediately after compression or during the storage period.

In some embodiments of the invention, dextrose is present in an amount from 55 to 95% by weight of the tablet. In some embodiments of the invention, dextrose is present in an amount from 60 to 95% by weight of the tablet. In some embodiments of the invention, dextrose is present in an amount from 65 to 95% by weight of the tablet. In some embodiments of the invention, dextrose is present in an amount from 70 to 95% by weight of the tablet. In some embodiments of the invention, dextrose is present in an amount from 70 to 90% by weight of the tablet.

In some embodiments of the invention, the oral chewable tablet is consisting essentially of dextrose, one or more active ingredients, one or more binders added separately in the formulation and being separate from any binder being integrated in other ingredients in the tablet, and auxiliary ingredients present up to about 5% by weight of the tablet.

In some embodiments of the invention, the oral chewable tablet is consisting essentially of dextrose, one or more active ingredients, and auxiliary ingredients present up to about 5% by weight of the tablet.

Flavors, high intensity sweeteners and glidant are examples of auxiliary ingredients that may be added in low amounts according to the invention without compromising the platform according to the invention.

However, in the present context, it is understood that a major part of the chewable tablets is composed of dextrose and the one or more active ingredients according to the invention. The overall system of the chewable tablet is controlled by these ingredients, including the special improved sensorial benefits of the invention, such as an improved mouth-feel, as well as saliva generation, and as well as friability properties.

In some embodiments of the invention, the oral chewable tablet is consisting essentially of dextrose, one or more active ingredients and one or more binders.

In some embodiments of the invention, the oral chewable tablet does not comprise sugar alcohol. It is contemplated that sugar alcohols in some embodiments are detrimental to the platform properties according to the invention, including sensorial properties, such as mouth-feel. In some embodiments, however, low amounts of sugar alcohol may be added, such as low amount of mannitol, such as less than 5% by weight.

In some embodiments of the invention, the oral chewable tablet does not comprise gum base. In some embodiments, the presence of gum base may impact the sensorial properties of the tablet as well as release of active ingredients.

In some embodiments of the invention, the dextrose is based on controlled enzymatic hydrolysis of starch.

In some embodiments of the invention, the dextrose comprises anhydrous dextrose. In some embodiments of the invention, the dextrose comprises hydrated dextrose. In some embodiments of the invention, the dextrose comprises dextrose monohydrate. In some embodiments of the invention, the dextrose comprises at least 90% dextrose equivalents calculated on a dry basis. In some embodiments of the invention, the dextrose comprises at least 93% dextrose equivalents calculated on a dry basis. In some embodiments of the invention, the dextrose comprises at least 95% dextrose equivalents calculated on a dry basis. In some embodiments of the invention, the dextrose comprises at least 98% dextrose equivalents calculated on a dry basis.

In some embodiments of the invention, the dextrose comprises a purified mixture of saccharides. In some embodiments of the invention, the dextrose comprises oligomeric saccharides. In some embodiments of the invention, the dextrose comprises 93 to 97% dextrose equivalents calculated on a dry basis. In some embodiments of the invention, the dextrose comprises microcrystalline dextrose. In some embodiments of the invention, the dextrose comprises dextrate.

In some embodiments of the invention, the dextrose does not comprise maltodextrin.

In some embodiments of the invention, the dextrose comprises 100% dextrose equivalents calculated on a dry basis. Dextrose in a pure version comprises 100% dextrose equivalents calculated on a dry basis and is presently preferred as the dextrose applied in the present invention. In some embodiments, dextrose is pure and is based on 100% conversion of starch to dextrose.

In some embodiments of the invention, the dextrose is directly compressible (DC).

In some embodiments of the invention, the dextrose is a powder.

In some embodiments of the invention, the oral chewable tablet comprises at least one grade of dextrose. In some embodiments of the invention, the oral chewable tablet comprises at least two grades of dextrose. In some embodiments of the invention, the oral chewable tablet comprises at least three grades of dextrose.

In some embodiments of the invention, the oral chewable tablet comprises different grades of dextrose in different layers of the tablet. In some embodiments of the invention, the oral chewable tablet comprises at least one grade of dextrose containing a binder. In some embodiments of the invention, the oral chewable tablet comprises at least one grade of dextrose containing maltodextrin. In some embodiments of the invention, the oral chewable tablet comprises at least one grade of dextrose being granules comprising dextrose and one or more binders. In some embodiments of the invention, the oral chewable tablet comprises at least one grade of dextrose without one or more binders being integrated in the grade.

In some embodiments of the invention, the dextrose comprises at least 30% by weight of particles in the range of 180 to 500 microns. In some embodiments of the invention, the dextrose comprises at least 50% by weight of particles in the range of 180 to 500 microns. In some embodiments of the invention, the dextrose comprises at least 30% by weight of particles in the range of 250 to 500 microns. In some embodiments of the invention, the dextrose comprises at least 50% by weight of particles above 250 microns. In some embodiments of the invention, the dextrose comprises at least 10% by weight of particles above 500 microns

In some embodiments of the invention, the dextrose comprises at most 35% by weight of particles below 100 microns. In some embodiments of the invention, the dextrose comprises at most 10% by weight of particles above 500 microns. One presently preferred grade of dextrose comprises C*Dex™ 02001 provided by Cargill. This grade is also referred to as a fine grade of dextrose due to the particle size distribution with a relatively high portion of particles with a small particles size. Another grade of dextrose comprises Cerelose® dextrose 020010 provided by Ingredion which has a particle size distribution similar to C*Dex™ 02001.

In some embodiments of the invention, the dextrose comprises at most 5% by weight of particles below 100 microns. In some embodiments of the invention, the dextrose comprises at most 20% by weight of particles below 250 microns. One grade of dextrose comprises C*Dex™ 02032 provided by Cargill. Another grade of dextrose comprises C*Dex™ 02030 provided by Cargill. This grade is also referred to as a coarse grade of dextrose due to the particle size distribution with a relatively higher portion of particles with a larger size.

In some embodiments of the invention, the dextrose comprises at most 25% by weight of particles below 149 microns. In some embodiments of the invention, the dextrose comprises at most 35% by weight of particles below 177 microns. In some embodiments of the invention, the dextrose comprises at least 5% by weight of particles above 1190 microns. One grade of dextrose comprises Royal-TR provided by Ingredion. This grade contain maltodextrin within the particles, i.e., the particles have been subject to granulation with maltodextrin.

In some embodiments, it was seen that the grade of dextrose may impact the compression configuration. For instance, by providing a coarse grade of dextrose certain challenges of capping or de-lamination may be avoided upon compression of the multi-layered tablets according to the invention. Accordingly, in some embodiments it is preferred with a more course grade of dextrose, or a grade with an integrated content of maltodextrin.

In some embodiments of the invention, the oral chewable tablet comprises one or more binders added separately in the formulation and being separate from any binder being integrated in other ingredients in the tablet.

In some embodiments of the invention, the one or more binders is present in an amount of 0.4 to 5% by weight of the tablet. In some embodiments of the invention, the one or more binders is present in an amount of 0.5 to 4% by weight of the tablet. In some embodiments of the invention, the one or more binders is present in an amount of 0.7 to 3% by weight of the tablet. In some embodiments of the invention, the one or more binders is present in an amount of 0.7 to 2% by weight of the tablet. In some embodiments of the invention, the one or more binders is present in an amount of 0.7 to 1.3% by weight of the tablet.

In some embodiments of the invention, the one or more binders is present in an amount of 2 to 15% by weight of the tablet. In some embodiments of the invention, the one or more binders is present in an amount of 3 to 10% by weight of the tablet.

In some embodiments of the invention, the one or more binders is present in an amount of 4 to 6% by weight of the tablet. In some embodiments of the invention, the one or more binders is present in an amount of 2 to 7% by weight of the tablet. In some embodiments of the invention, the one or more binders is present in an amount of 2 to 5% by weight of the tablet. In some embodiments of the invention, the one or more binders is present in an amount of 3 to 7% by weight of the tablet. In some embodiments of the invention, the one or more binders is present in an amount of 3 to 6% by weight of the tablet. In some embodiments of the invention, the one or more binders is present in an amount of 3 to 5% by weight of the tablet.

In some embodiments of the invention, the one or more binders is selected from the group consisting of hydroxypropyl cellulose (HPC), hydroxypropyl methylcellulose (HPMC), maltodextrin, and combinations thereof.

In some embodiments of the invention, the one or more binders is selected from the group consisting of hydroxypropyl cellulose (HPC), hydroxypropyl methylcellulose (HPMC), and combinations thereof.

In some embodiments of the invention, the one or more binders comprise hydroxypropyl cellulose (HPC). In some embodiments of the invention, the one or more binders is hydroxypropyl cellulose (HPC). HPC may be applied as a particular attractive binder. Thus, this binder, when used with dextrose, exhibits an advantageous sensory experience when compared to other well-known binders.

In some embodiments of the invention, the one or more binders comprise hydroxypropyl methylcellulose (HPMC). In some embodiments of the invention, the one or more binders is hydroxypropyl methylcellulose (HPMC). HPMC may be applied as a particular attractive binder. Thus, this binder, when used with dextrose, exhibits an advantageous sensory experience when compared to other well-known binders.

In some embodiments of the invention, the one or more binders comprises maltodextrin.

In some embodiments of the invention, the one or more binders does not comprise microcrystalline cellulose (MCC). It is contemplated that MCC is poor in relation to sensorial properties according to the invention.

In some embodiments of the invention, the one or more binders does not comprise silica, cellulose, silicified microcrystalline cellulose, clay, talc, starch, pregelatinized starch, calcium carbonate, dicalcium phosphate, magnesium carbonate, magnesium-alumino-metasilicates, hyper porous silica and mixtures thereof.

In some embodiments of the invention, the oral chewable tablet does not comprise one or more binders added separately in the formulation and being separate from any binder being integrated in other ingredients in the tablet. In some embodiments of the invention, the oral chewable tablet comprises one or more binders integrated in other ingredients in the tablet, such as one or more binders present in granules comprising dextrose.

In some embodiments of the invention, the one or more active ingredients is present in an amount of 5 to 50% by weight of the tablet.

In some embodiments of the invention, the one or more active ingredients is present in an amount of 10 to 50% by weight of the tablet.

In some embodiments of the invention, the one or more active ingredients is present in an amount of 20 to 50% by weight of the tablet.

In some embodiments of the invention, the one or more active ingredients is present in an amount of 30 to 50% by weight of the tablet.

In some embodiments of the invention, the one or more active ingredients is present in an amount of 5 to 40% by weight of the tablet.

In some embodiments of the invention, the one or more active ingredients is present in an amount of 5 to 30% by weight of the tablet.

In some embodiments of the invention, the one or more active ingredients is present in an amount of 10 to 30% by weight of the tablet.

In some embodiments of the invention, the one or more active ingredients comprise a non-directly compressible (non-DC) active ingredient.

In some embodiments of the invention, the one or more active ingredients is located in the first layer of the tablet. In some embodiments of the invention, the one or more active ingredients is located in the second layer of the tablet. In some embodiments of the invention, the one or more active ingredients is both located in the first and second layer of the tablet. In some embodiments of the invention, one active ingredient is located in the first and another active ingredient is located in the second layer of the tablet. In some embodiments of the invention, one of the layers of the tablet does not comprise the one or more active ingredients.

In some embodiments of the invention, the one or more active ingredients comprise a non-directly compressible (non-DC) active ingredient. In some embodiments of the invention, the one or more active ingredients comprise a non-directly compressible (non-DC) active ingredient in an amount of 5 to 50% by weight of the tablet. In some embodiments of the invention, the one or more active ingredients comprise a non-directly compressible (non-DC) active ingredient in an amount of 10 to 50% by weight of the tablet. In some embodiments of the invention, the one or more active ingredients comprise a non-directly compressible (non-DC) active ingredient in an amount of 20 to 50% by weight of the tablet. In some embodiments of the invention, the one or more active ingredients comprise a non-directly compressible (non-DC) active ingredient in an amount of 30 to 50% by weight of the tablet.

In some embodiments of the invention, the one or more active ingredients comprise a directly compressible (DC) active ingredient.

In some embodiments of the invention, the one or more active ingredients comprise an immune supporting active ingredient.

In some embodiments of the invention, the one or more active ingredients comprise a mixture of immune supporting active ingredients.

In some embodiments of the invention, the one or more active ingredients comprise an energy stimulating active ingredient

In some embodiments of the invention, the one or more active ingredients comprise a mixture of vitamins, minerals, and herbals.

In some embodiments of the invention, the one or more active ingredients comprise Vitamin C. In some embodiments of the invention, the one or more active ingredients comprise melatonin. In some embodiments of the invention, the one or more active ingredients comprise theanine. In some embodiments of the invention, the one or more active ingredients comprise calcium carbonate. In some embodiments of the invention, the one or more active ingredients comprise caffeine.

In some embodiments of the invention, the one or more active ingredients comprise multivitamin. In some embodiments of the invention, the one or more active ingredients comprise Zn-oxide. In some embodiments of the invention, the one or more active ingredients comprise Zn-citrate. In some embodiments of the invention, the one or more active ingredients comprise Zn-gluconate. In some embodiments of the invention, the one or more active ingredients comprise Vitamin D.

In some embodiments of the invention, the one or more active ingredients comprise acetaminophen. In some embodiments of the invention, the one or more active ingredients comprise phenylephrine. In some embodiments of the invention, the one or more active ingredients comprise dextromethorphan. In some embodiments of the invention, the one or more active ingredients comprise guaifenesin. In some embodiments of the invention, the one or more active ingredients comprise a combination of acetaminophen, phenylephrine, dextromethorphan, and guaifenesin.

In some embodiments of the invention, the one or more active ingredients comprise diphenhydramine. In some embodiments of the invention, the one or more active ingredients comprise loratadine. In some embodiments of the invention, the one or more active ingredients comprise nicotine.

In an embodiment of the invention the active ingredient is selected from acetylcysteine, ambroxol, amylmetacresol, benzocaine, bisacodyl, bismuth subsalicylate, bromhexine, cetirizine, dextromethorphan hydrobromide, 2,4-dichlorobenzyl alcohol, doxylamine succinate, flurbiprofen, glycerin, hexylresorcinol, lidocaine, menthol, myrrh, paracetamol, pectin, peppermint oil, phenol, phenylephrine hydrochloride, povidone-iodine, pseudoephedrine, ranitidine, simethicone, sodium docusate, spearmint, zinc, or any combination thereof.

The above list of active ingredients are active ingredients that may be delivered to the throat.

In some embodiments, the tablet may comprise further active ingredient, e.g. a combination of two or more active ingredients from the above list, or as a combination of an active ingredient from the above list and another active ingredient.

In an embodiment of the invention the active ingredient is an analgesic. Examples of analgesics include e.g. ibuprofen, paracetamol (acetaminophen), ketoprofen, aspirin (acetylsalicylic acid), and naproxen. In an embodiment of the invention the active ingredient is an anesthetic. In an embodiment of the invention the active ingredient is an anti-inflammation agent. In an embodiment of the invention the active ingredient is a disinfectant.

In an embodiment of the invention the active ingredient is a cough suppressant.

Examples of cough suppressants include e.g. dextromethorphan.

In an embodiment of the invention the active ingredient is an expectorant, such as guaifenesin.

In an embodiment of the invention the active ingredient is a local anesthetic. Examples of local anesthetics include e.g. ambroxol, benzocaine and hexylresorcinol.

In an embodiment of the invention the active ingredient is a member of the morphinan class. Examples of members of the morphinan class include e.g. dextromethorphan.

In an embodiment of the invention the active ingredient is a non-steroidal anti-inflammatory drug (NSAID). Examples of non-steroidal anti-inflammatory drugs (NSAIDs) include e.g. flurbiprofen.

In an embodiment of the invention the active ingredient is an anti-inflammation agent. In an embodiment of the invention the active ingredient is a disinfectant.

In an embodiment of the invention the active ingredient is a cough and cold agent.

In an embodiment of the invention the tablet comprises cough and cold agents including acetaminophen, dextromethorphan hydrobromide, guaifenesin and phenylephrine hydrochloride.

In an embodiment of the invention the tablet comprises cough and cold agents including acetaminophen, dextromethorphan hydrobromide and phenylephrine hydrochloride.

In an embodiment of the invention the active ingredient is an antihistamine. In an embodiment of the invention the active ingredient is an antibiotic. Examples of antibiotics include e.g. ampicillin, erythromycin, tetracycline, clarithromycin, penicillin, and metronidazole.

In an embodiment of the invention the active ingredient is an enzyme. One advantage of enzymes may be that digestion may be accelerated and/or that intestinal balance is restored or improved. In an embodiment of the invention the active ingredient is an opioid.

In an embodiment of the invention the tablet is a medical device for alleviating or treating dysphagia by inducing saliva generation. In an embodiment of the invention the active ingredient is cetirizine. In an embodiment of the invention the active ingredient is bromhexine. In an embodiment of the invention the active ingredient is amylmetacresol. In an embodiment of the invention the active ingredient is paracetamol. In an embodiment of the invention active ingredient is acetaminophen.

In an embodiment of the invention the active ingredient is dextromethorphan HBr. In an embodiment of the invention the active ingredient is guaifenesin. In an embodiment of the invention the active ingredient is phenylephrine HCl. In an embodiment of the invention the active ingredient is penicillin.

In an embodiment of the invention, the tablet further comprising a water-soluble fiber, such as inulin.

In an embodiment of the invention, the active ingredient comprises zinc gluconate and ascorbic acid. In an embodiment of the invention, the active ingredient comprises zinc.

In an embodiment of the invention, the tablet further comprising a plant extract, such as red clover or willow extract. In an embodiment of the invention, the tablet further comprising a plant extract, such as Echinacea, Camille or Lavender. In an embodiment of the invention, the plat extract is combined in the tablet with zinc gluconate and ascorbic acid.

In an advantageous embodiment of the invention the oral tablet is essentially consisting of ingredients that are present in nature.

In an advantageous embodiment of the invention the oral tablet comprises a natural high intensity sweetener, such as stevioside.

In some embodiments, the one or more active ingredients is selected from alginate, atenolol, aspirin (acetylsalicylic acid), ampicillin, aminosalicylates, anhydrous citric acid, aspirin, bisacodyl, bismuth subsalicylate, bupropion, caffeine, calcium, calcium carbonate, cetirizine, cimetidine, cisapride, clarithromycin, desloratadine, dexlansoprazole, diphenhydramine HCl, diphenhydramine citrate, dimenhydrinate, docusate erythromycin, dopamine, esomeprazole, famotidine, fexofenadine HCl, guaifenesin, hydrotalcite, ibuprofen, ketoprofen, lactase enzyme, lansoprazole, loratadine, lorcaserin, loperamide, loperamide HCl, magnesium, magnesium carbonate, magnesium hydroxide, melatonin, methamphetamine HCl, metoclopramide, metronidazole, montelukast, mycostatin, naltrexone, naproxen, naproxen sodium, nizatidine, omeprazole, ondansetron, orlistat, pantoprazole, paracetamol (acetaminophen), pectin, phentermine HCl, polypodium leucotomos, prednisolone, prednisone, progesterone, propranolol, propantheline bromide, pseudoephedrine HCl, phentermine, rabeprazole, ranitidine, roflumilast, scopoloamine butyl hydroxide, simethicone, sodium, sodium bicarbonate, sodium docusate, sumatriptan, testosterone, tetracycline, topiramate, vitamin A, vitamin B, vitamin B12, vitamin C (ascorbic acid), vitamin D, and vitamin E, vitamin K, prebiotics, probiotics, inulin fiber, citicoline, L-theanine, taurine, tryptophan, gamma-aminobutyric acid, or any combination thereof. In some embodiments of the invention, the active ingredient comprises L-theanine. In some embodiments of the invention, the active ingredient comprises GABA. In some embodiments of the invention, the active ingredient comprises bacopa. In some embodiments of the invention, the active ingredient comprises magnesium.

In some embodiments of the invention, the active ingredient comprises vitamin B. In some embodiments of the invention, the active ingredient comprises vitamin B3. In some embodiments of the invention, the active ingredient comprises vitamin B6. In some embodiments of the invention, the active ingredient comprises vitamin B12.

The above list of active ingredients are active ingredients that may be delivered to the gastrointestinal tract.

In some embodiments, the may comprise further active ingredient, e.g. a combination of two or more active ingredients from the above list, or as a combination of an active ingredient from the above list and another active ingredient.

Further ingredients include herbals, such as Ashwagandha, ginseng, elderberry, Boswellia, green tea, green coffee bean extract, coffee fruit extract, willow bark, Ivy leaf, rose hip, chamonile, forsythia fruit extract, lemon balm, passionflower extract, zembrin, and root of marshmallow. In some embodiments, the active ingredient comprises ginseng.

In an advantageous embodiment of the invention the active ingredient is an analgesic. Examples of analgesics include e.g. ibuprofen, paracetamol (acetaminophen), ketoprofen, aspirin (acetylsalicylic acid), and naproxen. In an advantageous embodiment of the invention the active ingredient is an anesthetic. In an advantageous embodiment of the invention the active ingredient is an anti-inflammation agent. In an advantageous embodiment of the invention the active ingredient is a disinfectant. In an advantageous embodiment of the invention the active ingredient is an antibiotic. Examples of antibiotics include e.g. ampicillin, erythromycin, tetracycline, clarithromycin, penicillin, and metronidazole.

In an advantageous embodiment of the invention the active ingredient is selected from vitamins, minerals, and supplements (VMS).

Examples of vitamins, minerals, and supplements include e.g. vitamin A, vitamin B, vitamin B12, vitamin C, vitamin D, vitamin E, vitamin K.

In some embodiments of the invention, the tablet comprises a combination of caffeine, L-theanine, vitamin B3, vitamin B6, and vitamin B12. In some embodiments of the invention, the tablet comprises a combination of caffeine, and vitamin B6. In some embodiments of the invention, the tablet comprises a combination of ginseng, and vitamin B12. In some embodiments of the invention, the tablet comprises a combination of melatonin, vitamin C, and zinc. In some embodiments of the invention, the tablet comprises a combination of L-theanine, and GABA. In some embodiments of the invention, the tablet comprises a combination of L-theanine, and bacopa.

In an advantageous embodiment of the invention the active ingredient is a hormone. In an advantageous embodiment of the invention the active ingredient is melatonin. Examples of hormones include e.g. progesterone, testosterone, and melatonin. In an advantageous embodiment of the invention the active ingredient is a steroid. Examples of steroids include e.g. prednisolone and prednisone. In an advantageous embodiment of the invention the active ingredient is a proton pump inhibitor. Examples of proton pump inhibitors include e.g. rabeprazole, pantoprazole, esomeprazole, dexlansoprazole, lansoprazole, and omeprazole.

In an advantageous embodiment of the invention the active ingredient is an antihistamine. Examples of antihistamines include e.g. cimetidine, ranitidine, famotidine, nizatidine, and desloratadine. Antihistamines are drugs to treat allergic rhinitis and other allergies. Antihistamines can give release to a person with nasal congestion, sneezing or hives caused by e.g. pollen, dust mites or animal allergy.

In an advantageous embodiment of the invention the active ingredient is a triptan. Examples of triptans include e.g. sumatriptan.

In an advantageous embodiment of the invention the active ingredient is a xerostomia mitigation agent, such as a xerostomia mitigation agent for cancer patients. In an advantageous embodiment of the invention the active ingredient is a migraine treatment agent. In an advantageous embodiment of the invention the active ingredient is an enzyme.

In an advantageous embodiment of the invention the active ingredient is a probiotic ingredient. In an advantageous embodiment of the invention the active ingredient is a prebiotic ingredient.

In an advantageous embodiment of the invention the active ingredient is a gastrointestinal medication. In this context a gastrointestinal medication is understood as an active ingredient acting in the gastrointestinal tract.

In an advantageous embodiment of the invention the active ingredient is an opioid. In an advantageous embodiment of the invention the active ingredient is an allergy medication. In an advantageous embodiment of the invention the active ingredient is loratadine. In an advantageous embodiment of the invention the active ingredient is diphenhydramine.

In an advantageous embodiment of the invention the tablet is a medical device for alleviating or treating dysphagia by inducing saliva generation.

In an advantageous embodiment of the invention the active ingredient is ampicillin. In an advantageous embodiment of the invention the active ingredient is ibuprofen. In an advantageous embodiment of the invention the active ingredient is ondansetron. In an advantageous embodiment of the invention the active ingredient is paracetamol (acetaminophen). In an advantageous embodiment of the invention the active ingredient is acetylsalicylic acid. In an advantageous embodiment of the invention the active ingredient is simethicone. In an advantageous embodiment of the invention the active ingredient is sodium docusate.

In some embodiments of the invention, the one or more active ingredients comprise an active pharmaceutical ingredient.

In some embodiments of the invention, friability of the tablet is less than 3%, such as less than 2%, such as less than 1.5%, wherein friability is measured according to European Pharmacopoeia 9.1, test method 2.9.7. by using a pharmaceutical friability-tester PTF 10E from Pharma Test.

In some embodiments of the invention, the tablet generates more than 1.5 mL saliva within 30 seconds from onset of mastication. In some embodiments of the invention, the tablet generates more than 1.5 mL saliva within a period from 30 to 90 seconds from onset of mastication. In some embodiments of the invention, the tablet generates more than 1.5 mL saliva within a period from 90 to 180 seconds from onset of mastication. In some embodiments of the invention, the tablet generates more than 1.5 mL saliva within a period from 180 to 300 seconds from onset of mastication. In some embodiments of the invention, the oral chewable tablet further comprises a saliva production inhibiting agent for controlling saliva production.

In some embodiments of the invention, the oral chewable tablet is designed to release the active ingredient in the oral cavity and designed to deliver a part of the active ingredient to the throat as part of saliva generated upon mastication of the tablet. In some embodiments of the invention, the oral chewable tablet is designed to release the active ingredient in the oral cavity and designed to deliver a part of the active ingredient to the gastrointestinal tract as part of saliva generated upon mastication of the tablet. In some embodiments of the invention, oral chewable tablet comprises means for accelerated release of the one or more active ingredient.

In some embodiments of the invention, the oral chewable tablet comprises one or more disintegrants operable to disintegrate the tablet within a period of 2 minutes or less in contact with oral saliva.

In the present context, “disintegrated” or “disintegrate” is intended to mean that the tablet is no longer to be considered a tablet but the tablet has been reduced and/or dispersed in saliva.

Specifically, the content of disintegrants greatly facilitate disintegration of the tablet according to the invention. However, while disintegrants have previously been used in tablet formulation science, the particular combination of disintegrants with dextrose according to the application would have been seen as problematic in view of the specific properties of dextrose. Various problems were suspected by the inventors of the present application, such as sensorial drawbacks and concentration issues with a high load of the active ingredients.

In some embodiments of the invention, the oral chewable tablet comprises one or more disintegrants selected from the group consisting of sodium croscarmellose, crospovidone, sodium starch glycolate, and combinations thereof.

In an embodiment of the invention, the one or more disintegrants comprises cross-linked polyvinylpyrrolidone.

In an embodiment of the invention, the one or more disintegrants comprises cross-linked polyvinylpyrrolidone and wherein at least 50% by weight of the cross-linked polyvinylpyrrolidone has a particle size below 50 micrometers.

In an embodiment of the invention, the one or more disintegrants comprises cross-linked polyvinylpyrrolidone and wherein at least 25% by weight of the cross-linked polyvinylpyrrolidone has a particle size below 15 micrometers.

In some embodiments of the invention, the first layer is compressed under a compression pressure of 1 to 5 kN. In some embodiments of the invention, the second layer is compressed under a compression pressure of 6 to 40 kN. In some embodiments of the invention, the second layer is compressed under a compression pressure of 10 to 30 kN.

In some embodiments of the invention, the oral chewable tablet in contact with saliva has a disintegration profile that varies less than 10% under a compression pressure of to 30 kN.

In some embodiments of the invention, the unit weight of the tablet is from about 100 mg to about 2000 mg. In some embodiments of the invention, the unit weight of the tablet is from about 100 mg to about 1800 mg. In some embodiments of the invention, the unit weight of the tablet is from about 500 mg to about 1600 mg. In some embodiments of the invention, the unit weight of the tablet is from about 600 mg to about 1500 mg.

In some embodiments of the invention, wherein the one or more active ingredients are present in an amount of 1 to 1000 mg. In some embodiments of the invention, wherein the one or more active ingredients are present in an amount of 1 to 800 mg.

In some embodiments of the invention, the one or more active ingredients are present in an amount of 1 to 600 mg.

In some embodiments of the invention, the one or more active ingredients are present in an amount of 50 to 250 mg. In some embodiments of the invention, the one or more active ingredients are present in an amount of 100 to 250 mg.

In some embodiments of the invention, the one or more active ingredients are present in an amount of 1 to 50 mg. In some embodiments of the invention, the one or more active ingredients are present in an amount of 1 to 40 mg. In some embodiments of the invention, the one or more active ingredients are present in an amount of 1 to 30 mg. In some embodiments of the invention, the one or more active ingredients are present in an amount of 1 to 20 mg. In some embodiments of the invention, the one or more active ingredients are present in an amount of 1 to 10 mg. In some embodiments of the invention, the one or more active ingredients are present in an amount of 1 to 5 mg. In some embodiments of the invention, the one or more active ingredients are present in an amount of 1 to 4 mg.

In some embodiments of the invention, the oral chewable tablet provides an improved mouthfeel compared to an oral chewable tablet comprising less than 50% by weight of dextrose.

In some embodiments of the invention, the oral chewable tablet provides an improved melting sensation compared to an oral chewable tablet comprising less than 50% by weight of dextrose.

In some embodiments of the invention, the oral chewable tablet provides an improved liquid sensation compared to an oral chewable tablet comprising less than 50% by weight of dextrose.

In some embodiments of the invention, the oral chewable tablet provides a less stickiness sensation compared to an oral chewable tablet comprising less than 50% by weight of dextrose.

In some embodiments of the invention, the oral chewable tablet provides a less bitterness sensation from the one or more active ingredients compared to an oral chewable tablet comprising less than 50% by weight of dextrose.

In some embodiments of the invention, the oral chewable tablet provides improved taste masking compared to an oral chewable tablet comprising less than 50% by weight of dextrose.

In some embodiments of the invention, the oral chewable tablet provides an improved melting sensation compared to an oral chewable tablet comprising one or more separate binders.

In some embodiments of the invention, the oral chewable tablet is designed to turn into liquid within 60 seconds of mastication.

In some embodiments of the invention, the oral chewable tablet is designed to turn into liquid within 30 seconds of mastication.

In some embodiments of the invention, the oral chewable tablet is designed to turn into liquid within 15 seconds of mastication.

In an embodiment of the invention, the dextrose is evenly distributed in the tablet or at least one module of the tablet.

One advantage of the above embodiment may be that the even distribution of the dextrose promotes an effective disintegration of the module upon mastication, e.g., due to lower mechanical strength contribution from the dextrose, thereby facilitating effective contacting of the resulting mastication fragments formed by the mastication with saliva, again increasing dissolving of the tablet. Also, the even distribution of the dextrose promotes a high number of mastication fragments with dextrose, which again effectively promotes salivation. Thus, a synergy between utilization of dextrose as a disintegration promoter due to the lower mechanical strength and also as a salivation promoter in combination with the even distribution to facilitate effect dispersion of mastication fragments in the oral cavity upon mastication.

In some embodiments of the invention, the oral chewable tablet comprises a further tablet module with a different disintegration time.

In an embodiment of the invention, the resistance to crunching of the tablet is greater than 60N, such as greater than 70N, such as greater than 80N, such as greater than 90N, such as greater than 100 N, such as greater than 110, such as greater than 130N such as greater than 150N, wherein the resistance to crunching of the tablet is less than 300N, such as less than 250N, such as less than 200N, wherein the resistance to crunching is determined according to European Pharmacopoeia 9.1, test method 2.9.8. by using a pharmaceutical resistance to crunching tester model Pharma Test type PTB 311.

High intensity artificial sweetening agents can also be used alone or in combination with the above dextrose. Preferred high intensity sweeteners include, but are not limited to sucralose, aspartame, salts of acesulfame, alitame, saccharin and its salts, cyclamic acid and its salts, glycyrrhizin, dihydrochalcones, thaumatin, monellin, stevioside (natural intensity sweetener) and the like, alone or in combination. In order to provide longer lasting sweetness and flavor perception, it may be desirable to encapsulate or otherwise control the release of at least a portion of the artificial sweeteners. Techniques such as wet granulation, wax granulation, spray drying, spray chilling, fluid bed coating, conservation, encapsulation in yeast cells and fiber extrusion may be used to achieve desired release characteristics. Encapsulation of sweetening agents can also be provided using another tablet component such as a resinous compound.

Usage level of the artificial sweetener will vary considerably and will depend on factors such as potency of the sweetener, rate of release, desired sweetness of the product, level and type of flavor used and cost considerations. Thus, the active level of artificial sweetener may vary from about 0.001 to about 3% by weight (preferably from about 0.02 to about 3% by weight). When carriers used for encapsulation are included, the usage level of the encapsulated sweetener will be proportionately higher. Combinations of sugar and/or non-sugar sweeteners may be used in the formulation.

In an embodiment of the invention, the tablet comprises flavor.

The amount of flavor may e.g., be from 0.1 to about 5% by weight of the tablet, such as 0.1 to about 3% by weight of the tablet.

Usable flavors include almond, almond amaretto, apple, Bavarian cream, black cherry, black sesame seed, blueberry, brown sugar, bubblegum, butterscotch, cappuccino, caramel, caramel cappuccino, cheesecake (graham crust), chili, cinnamon redhots, cotton candy, circus cotton candy, clove, coconut, coffee, clear coffee, double chocolate, energy cow, ginger, glutamate, graham cracker, grape juice, green apple, Hawaiian punch, honey, Jamaican rum, Kentucky bourbon, kiwi, koolada, lemon, lemon lime, tobacco, maple syrup, maraschino cherry, marshmallow, menthol, milk chocolate, mocha, Mountain Dew, peanut butter, pecan, peppermint, raspberry, banana, ripe banana, root beer, RY 4, spearmint, strawberry, sweet cream, sweet tarts, sweetener, toasted almond, tobacco, tobacco blend, vanilla bean ice cream, vanilla cupcake, vanilla swirl, vanillin, waffle, Belgian waffle, watermelon, whipped cream, white chocolate, wintergreen, amaretto, banana cream, black walnut, blackberry, butter, butter rum, cherry, chocolate hazelnut, cinnamon roll, cola, creme de menthe, eggnog, English toffee, guava, lemonade, licorice, maple, mint chocolate chip, orange cream, peach, pina colada, pineapple, plum, pomegranate, pralines and cream, red licorice, salt water taffy, strawberry banana, strawberry kiwi, tropical punch, tutti frutti, vanilla, or any combination thereof.

### DETAILED DESCRIPTION OF THE INVENTION

The invention will now be described in more details with respect to certain aspects and embodiments of the invention. These aspects and embodiments are intended to be understood in connection with the rest of the description, including the Summary of the Invention and the Examples of the invention.

The verb “to comprise” as is used in this description and in the claims and its conjugations are used in its non-limiting sense to mean that items following the word are included, but items not specifically mentioned are not excluded. In addition, reference to an element by the indefinite article “a” or “an” does not exclude the possibility that more than one of the elements are present, unless the context clearly requires that there is one and only one of the elements. The indefinite article “a” or “an” thus usually means “at least one”. Additionally, the words “a” and “an” when used in the present document in connection with the word comprising or containing denote “one or more.” The expression “one or more” is intended to mean one, two, three or more.

As used herein, the term “approximately” or “about” in reference to a number are generally taken to include numbers that fall within a range of 5%, 10%, 15%, or 20% in either direction (greater than or less than) of the number unless otherwise stated or otherwise evident from the context (except where such number would be less than 0% or exceed 100% of a possible value).

As used herein the term “oral chewable tablet” is considered a tablet for oral use. Particularly, the oral tablet is considered as formed by tableting, i.e., compression of a particle composition, comprising a population of particles. Thus, the tablet is considered a compressed tablet formed by a plurality of particles. Typically, the oral chewable tablet may also be referred to as a tablet or oral tablet.

The term “particle size” relates to the ability of the particles to move through or be retained by sieve holes of a specific size. As used herein, the term “particle size” refers to the average particle size as determined according to European Pharmacopoeia 9.1 when using test method 2.9.38 particle size distribution estimation by analytical sieving, unless otherwise specifically is mentioned.

The term “particle” or similar wording is intended to denote a single, discrete composition of solid matter, such as a granule or individual elements in powder, having a certain size that may deviate considerable.

The term “weight of the oral tablet” or similar wording meaning the same is defined in the present context as weight of the oral tablet, not including the weight of an outer coating, such as a hard coating, soft coating, and the like.

By the phrase “texture” is meant a qualitative measure of the properties of the oral tablet and of the overall mouth-feel experienced by the user during use. Thus, the term “texture” encompasses measurable quantities such as hardness as well as more subjective parameters related to the feel experienced by a user.

The term “release” in the present context is intended to mean under “in vitro” conditions if not stated otherwise. In particular, the “release rate” during a certain period of time is intended to mean the amount in percentage of active ingredients that is released during the period. In the present context the term “release” refers to the released substance being liberated from the water-soluble matrix. In some embodiments, the process of releasing a substance corresponds to the substance being dissolved in saliva.

The term “sustained release” or “extended release” is herein intended to mean prolonged release over time. The term “rapid release” or “quick release” or “high release” is herein intended to mean a higher content released for a given period of time. The term “controlled release” is intended to mean a release of a substance from an oral tablet by the aid of active use of the oral tablet in the oral cavity of the subject, whereby the active use is controlling the amount of substance released.

In the present context the term “turn into liquid” is intended to mean that the tablet disintegrates and the fragments or particles of the tablet are either suspended or dissolved in saliva, perceived as liquid by a test person.

As used herein, the term “disintegrate” refers to a reduction of an object to components, fragments or particles. Disintegration time may be measured in vitro or in vivo. Unless otherwise stated, the in vitro measurements are carried out in accordance to European Pharmacopeia 9.0, section 2.9.1, Disintegration of tablets and capsules.

As used herein, the term “dissolve” is the process where a solid substance enters a solvent (oral saliva) to yield a solution. Unless otherwise stated, dissolving implies a full dissolving of the compound in question.

As used herein, the terms “disintegrant” refers to an ingredient facilitating disintegration of an FDT-module, when the FDT-module comes into contact with saliva. Disintegrants usable within the scope of the invention may include starch, pregelatinated starch, modified starch (including potato starch, maize starch, starch 1500, sodium starch glycolate and starch derivatives), cellulose, microcrystalline cellulose, alginates, and superdisintegrants, such as crosslinked cellulose (such as sodium carboxy methyl cellulose), crosslinked polyvinyl pyrrolidone (PVP), crosslinked starch, crosslinked alginic acid, natural superdisintegrants, and calcium silicate. Disintegrants may often be considered as measure promoting the break-up of the module into smaller fragments upon administration to facilitate nicotine release and eventual absorption. Crospovidone may comprise various grades, such as Kollidon CL-F or Kollidon CL-SF available from BASF.

When referring to induced saliva generation, it is noted that this induced saliva generation exceeds any saliva generation without the use of the tablet of the invention, or with a content of less than 50% dextrose. Particularly, in an embodiment, the induced saliva generation exceeds saliva generation when using conventional tablets without dextrose or with less than 50% dextrose. Then, induced saliva generation is increased over any saliva generation associated with conventional products, e.g. by comparing with a tablet without dextrose or with less than 50% dextrose.

When referring to induced saliva generation, the saliva generation may be tested using the following method:

Test subject abstain from eating and drinking at least 30 minutes before initiation of any test. Immediately before introducing of the tablet into the oral cavity, the test subject swallows. The test subject refrains from swallowing during the test. Immediately after introducing of the tablet into the oral cavity, the test subject starts masticating the tablet at a frequency of 1 chew per second for 20 seconds. Then, saliva and any remains of the tablet is kept in the mouth within chewing for 10 second. 30 seconds after starting the test, the test subject discards saliva including any tablet fragments into a plastic cup, which is weighted. Saliva discarded also at 90 seconds after onset of mastication, at 180 seconds after onset of mastication, at 300 seconds after onset of mastication, at 420 seconds after onset of mastication, and at 600 seconds after onset of mastication. At all times, the test subject makes as little movement as possible, and refrains from swallowing.

As used herein the term “active ingredient” refers to a substance that is biologically active and has a physiological effect on the human body for the benefit of the human body or part thereof. Active ingredients include active pharmaceutical ingredients, but also other active substances such as nutraceuticals or immune supporting active ingredients.

In the following raw materials will refer to the mixed particles to be compressed into a tablet according to embodiments of the invention unless otherwise stated.

The following description outlines explanations of how the tablet of the invention may be produced and further details of what may be added to the inventive composition.

Typically, the process of manufacture of the inventive tablet may be performed in a single tablet press, such as a rotary tablet press. But it may be a benefit under some circumstances to apply a separate tablet press.

Preferably, the upper punch is convex which gives the upper face of the pressed tablet a concave form.

It should of course be noted that the shape of the punches may vary depending on the desired tablet shape.

In some embodiments of the invention, pressing of the tablets are performed at a force of 10 to 50 kN. In some embodiments of the invention, pressing of the tablets are performed at a force of 10 to 40 kN. In some embodiments of the invention, pressing of the tablets are performed at a force of 10 to 30 kN.

The oral tablet according to the invention is manufactured by applying pressure to a content of particles by suitable compression means. The particles or powder is then pressed into a compact coherent tablet. The particles may for example comprise so-called primary particles or aggregated primary particles. When these are pressed, bonds are established between the particles or granules, thereby conferring a certain mechanical strength to the pressed tablet.

It should be noted that the above-introduced terms: powder, primary particles and aggregated primary particles may be somewhat misleading in the sense that the difference between primary particles and aggregated primary particles may very often be looked upon differently depending on the background of the user. Some may for instance regard a sweetener as a primary particle in spite of the fact that this particle due to the typically preprocessing performed when delivered to the customer should rather be regarded as some sort of aggregated primary particles. The definition adopted in the description of this invention is that aggregated primary particles refer to macro-particles comprising more or less preprocessed primary particles.

When pressure is applied to the particles, the bulk volume is reduced, and the amount of air is decreased. During this process energy is consumed. As the particles come into closer proximity to each other during the volume reduction process, bonds may be established between the particles or granules. The formation of bonds is associated with a reduction in the energy of the system as energy is released. Volume reduction takes place by various mechanisms and different types of bonds may be established between the particles or granules depending on the pressure applied and the properties of the particles or granules. The first thing that happens when a powder is pressed is that the particles are rearranged under low compaction pressures to form a closer packing structure. Particles with a regular shape appear to undergo rearrangement more easily than those of irregular shape. As the pressure increases, further rearrangement is prevented, and subsequent volume reduction is obtained by plastic and elastic deformation and/or fragmentation of the tablet particles. Brittle particles are likely to undergo fragmentation, i.e. breakage of the original particles into smaller units. Plastic deformation is an irreversible process resulting in a permanent change of particle shape, whereas the particles resume their original shape after elastic deformation. Evidently, both plastic and elastic deformation may occur, when compressing an oral tablet.

Several studies of the bond types in pressed tablets have been made over the years, typically in the context of pharmaceuticals and several techniques of obtaining pressed tablets on the basis of available powders has been provided. Such studies have been quite focused on what happens when the volume reduction is performed and how the end-product may be optimized for the given purpose. Several refinements with respect to pressed tablets has for instance been made in the addition of for example binders in the tablet raw materials for the purpose of obtaining a sufficient strength to the final pressed tablet while maintaining acceptable properties, e.g. with respect to release.

By the method of the invention, it is possible to form one-layered or multi-layered tablets, such as two-layered tablets or three-layered tablets.

In accordance with the invention, the tableted oral tablet according to the invention may comprise about 0.1 to about 75% by weight of an outer coating applied onto the oral tablet centre. Thus, suitable coating types include hard coatings, film coatings and soft coatings of any composition including those currently used in coating of tableted oral tablet.

One presently preferred outer coating type is a hard coating, which term is used in the conventional meaning of that term including sugar coatings and sugar-free (or sugarless) coatings and combinations thereof. The object of hard coating is to obtain a sweet, crunchy layer, which is appreciated by the consumer and it may moreover protect the oral tablet centres for various reasons. In a typical process of providing the oral tablet centres with a protective sugar coating, the oral tablet centres are successively treated in suitable coating equipment with aqueous solutions of crystallisable sugar such as sucrose or dextrose, which, depending on the stage of coating reached, may contain other functional ingredients, e.g. fillers, binding agents, colours, etc. In the present context, the sugar coating may contain further functional or active compounds including flavour compounds and/or active compounds.

In a typical hard coating process as it will be described in detail in the following, a suspension containing crystallisable sugar and/or polyol is applied onto the oral tablet centres and the water it contains is evaporated off by blowing with air. This cycle must be repeated several times, typically 3 to 80 times, in order to reach the swelling required. The term “swelling” refers to the increase in weight or thickness of the products, as considered at the end of the coating operation by comparison with the beginning, and in relation to the final weight or thickness of the coated products. In accordance with the present invention, the coating layer constitutes about 0.1 to about 75% by weight of the finished oral tablet element, such as about 10 to about 60% by weight, including about 15 to about 50% by weight.

In further useful embodiments, the outer coating of the oral tablet element of the invention is an element that is subjected to a film coating process and which therefore comprises one or more film-forming polymeric agents and optionally one or more auxiliary compounds, e.g. plasticizers, pigments and opacifiers. A film coating is a thin polymer-based coating applied to an oral tablet centre of any of the above forms. The thickness of such a coating is usually between 20 and 100 μm.

Generally, the film coating is obtained by passing the oral tablet centres through a spray zone with atomized droplets of the coating materials in a suitable aqueous or organic solvent vehicle, after which the material adhering to the oral tablet centres is dried before the next portion of coating is received. This cycle is repeated until the coating is complete.

In one embodiment the tablet according to the invention comprises a pharmaceutically, cosmetically or biologically active substance. Examples of such active substances, a comprehensive list of which is found e.g. in WO 00/25598, which is incorporated herein by reference, include drugs, dietary supplements, antiseptic agents, pH adjusting agents, anti-smoking agents. Examples of useful active substances in the form of antiseptics include salts and derivatives of guanidine and biguanidine the following types of substances with limited water-solubility: quaternary ammonium compounds (e.g. ceramine, chloroxylenol, crystal violet, chloramine), aldehydes (e.g. paraformaldehyde), derivatives of dequaline, polynoxyline, phenols (e.g. thymol, p-chlorophenol, cresol), hexachlorophene, salicylic anilide compounds, triclosan, halogenes (iodine, iodophores, chloroamine, dichlorocyanuric acid salts), alcohols (3,4 dichlorobenzyl alcohol, benzyl alcohol, phenoxyethanol, phenylethanol), cf. also Martindale, The Extra Pharmacopoeia, 28th edition, pages 547-578; metal salts, complexes and compounds with limited water-solubility, such as aluminum salts, (for instance aluminum potassium sulphate AIK (SO4) 2, 12H2O) and salts, complexes and compounds of boron, barium, strontium, iron, calcium, zinc, (zinc acetate, zinc chloride, zinc gluconate), copper (copper chloride, copper sulphate), lead, silver, magnesium, sodium, potassium, lithium, molybdenum, vanadium should be included.

Examples of active substances in the form of agents adjusting the pH in the oral cavity include: acids, such as adipic acid, succinic acid, fumaric acid, or salts thereof or salts of citric acid, tartaric acid, malic acid, acetic acid, lactic acid, phosphoric acid and glutaric acid and acceptable bases, such as carbonates, hydrogen carbonates, phosphates, sulphates or oxides of sodium, potassium, ammonium, magnesium or calcium, especially magnesium and calcium.

Active ingredients may comprise the below mentioned compounds or derivates thereof but are not limited thereto: Acetaminophen, Acetylsalicylic acid, Buprenorphine, Bromhexin, Celcoxib, Codeine, Diphenhydramineiclofenac, Etoricoxib, Ibuprofen, Indometacin, Ketoprofen, Lumiracoxib, Morphine, Naproxen, Oxycodon, Parecoxib, Piroxicam, Pseudoefedrin, Rofecoxib, Tenoxicam, Tramadol, Valdecoxib, Calciumcarbonat, Magaldrate, Disulfiram, Bupropion, Nicotine, Azithromycin, Clarithromycin, Clotrimazole, Erythromycin, Tetracycline, Granisetron, Ondansetrone, Prometazin, Tropisetron, Brompheniramine, Ceterizin, leco-Ceterizin, Chlorcyclizine, Chlorpheniramin, Chlorpheniramin, Difenhydramine, Doxylamine, Fenofenadin, Guaifenesin, Loratidin, des-Loratidin, Phenyltoloxamine, Promethazin, Pyridamine, Terfenadin, Troxerutin, Methyldopa, Methylphenidate, Benzalcon. Chloride, Benzeth. Chloride, Chloride, Ecabet-sodium, Haloperidol,

Allopurinol, Colchinine, Theophylline, Propanolol, Prednisolone, Prednisone, Urea, Actot, Glibenclamide, Glipizide, Metformin, Miglitol, Repaglinide, Rosiglitazone,

Apomorfin, Cialis, Sildenafil, Vardenafil, Diphenoxylate, Simethicone, Cimetidine, Famotidine, Ranitidine, Ratinidine, cetrizin, Loratadine, Aspirin, Benzocaine, Dextrometorphan, Phenylpropanolamine, Pseudoephedrine, Cisapride, Domperidone, Metoclopramide, Acyclovir, Dioctylsulfosucc., Phenolphtalein, Almotriptan, Eletriptan, Ergotamine, Migea, Naratriptan, Rizatriptan, Sumatriptan, Zolmitriptan, Aluminum salts, Calcium salts, Ferro salts, Ag-salts, Zinc-salts, Amphotericin B, Miconazole, Triamcinolonacetonid, Melatonine, Phenobarbital, Caffeine, Benzodiazepiner, Hydroxyzine, Meprobamate, Phenothiazine, Buclizine, Brometazine, Cinnarizine, Cyclizine, Difenhydramine, Dimenhydrinate, Buflomedil, Amphetamine, Caffeine, Ephedrine, Orlistat, Phenylephedrine, Phenylpropanolamin, Pseudoephedrine, Sibutramin, Ketoconazole, Nitroglycerin, Nystatin, Progesterone, Testosterone, Vitamin B12, Vitamin C, Vitamin A, Vitamin D, Vitamin E, Pilocarpin, Aluminumaminoacetat, Cimetidine, Esomeprazole, Famotidine, Lansoprazole, Magnesiumoxide, Nizatide and or Ratinidine.

The invention is suitable for increased or accelerated release of active agents selected among the group of dietary supplements, antiseptic agents, pH adjusting agents, anti-smoking agents, sweeteners, flavorings, aroma agents or drugs. Some of those will be described below.

The active agents to be used in connection with the present invention may be any substance desired to be released from the tablet. The active agents, for which a controlled and/or accelerated rate of release is desired, are primarily substances with a limited water-solubility, typically below 10 g/100 mL inclusive of substances which are totally water-insoluble. Examples are medicines, dietary supplements, oral compositions, anti-smoking agents, highly potent sweeteners, pH adjusting agents, flavorings etc.

Other active ingredients are, for instance, paracetamol, benzocaine, cinnarizine, menthol, carvone, caffeine, cyclizine hydrochloride, 1,8-cineol, nandrolone, miconazole, mystatine, nicotine, other quaternary ammonium compounds, vitamin E, vitamin A, vitamin D, glibenclamide or derivatives thereof, progesterone, acetylsalicylic acid, dimenhydrinate, cyclizine, metronidazole, sodium hydrogen carbonate, the active components from ginkgo, the active components from propolis, the active components from ginseng, methadone, oil of peppermint, salicylamide, hydrocortisone or astemizole.

Examples of active agents in the form of dietary supplements are for instance salts and compounds having the nutritive effect of vitamin B2 (riboflavin), B12, folinic acid, folic acid, niacine, biotine, poorly soluble glycerophosphates, amino acids, the vitamins A, D, E and K, minerals in the form of salts, complexes and compounds containing calcium, phosphorus, magnesium, iron, zinc, copper, iodine, manganese, chromium, selenium, molybdenum, potassium, sodium or cobalt.

Furthermore, reference is made to lists of nutritionists accepted by the authorities in different countries such as for instance US code of Federal Regulations, Title 21, Section 182.5013.182 5997 and 182.8013-182.8997.

Examples of active agents in the form of antiseptics are for instance salts and compounds of guanidine and biguanidine and the following types of substances with limited water-solubility: quaternary ammonium compounds (for instance ceramine, chloroxylenol, crystal violet, chloramine), aldehydes (for instance paraformaldehyde), compounds of dequaline, polynoxyline, phenols (for instance thymol, para chlorophenol, cresol) hexachlorophene, salicylic anilide compounds, triclosan, halogenes (iodine, iodophores, chloroamine, dichlorocyanuric acid salts), alcohols (3,4 dichlorobenzyl alcohol, benzyl alcohol, phenoxyethanol, phenylethanol), cf. furthermore Martindale, The Extra Pharmacopoeia, 28th edition, pages 547-578; metal salts, complexes and compounds with limited water-solubility, such as aluminum salts, (for instance aluminum potassium sulphate AIK (SO4) 2,12H2O) and furthermore salts, complexes and compounds of boron, barium, strontium, iron, calcium, zinc, (zinc acetate, zinc chloride, zinc gluconate), copper (copper chloride, copper sulfate), lead, silver, magnesium, sodium, potassium, lithium, molybdenum, vanadium should be included.

Examples of active agents in the form of agents adjusting the pH in the oral cavity include for instance: acceptable acids, such as adipic acid, succinic acid, fumaric acid, or salts thereof or salts of citric acid, tartaric acid, malic acid, acetic acid, lactic acid, phosphoric acid and glutaric acid and acceptable bases, such as carbonates, hydrogen carbonates, phosphates, sulfates or oxides of sodium, potassium, ammonium, magnesium or calcium, especially magnesium and calcium.

Examples of active agents in the form of anti-smoking agents include for instance: nicotine, tobacco powder or silver salts, for instance silver acetate, silver carbonate and silver nitrate.

Further examples of active agents are medicines of any type.

Examples of active agents in the form of medicines include caffeine, salicylic acid, salicyl amide and related substances (acetylsalicylic acid, choline salicylate, magnesium salicylate, sodium salicylate), paracetamol, salts of pentazocine (pentazocine hydrochloride and pentazocinelactate), buprenorphine hydrochloride, codeine hydrochloride and codeine phosphate, morphine and morphine salts (hydrochloride, sulfate, tartrate), methadone hydrochloride, ketobemidone and salts of ketobemidone (hydrochloride), beta-blockers, (propranolol), calcium antagonists, verapamil hydrochloride, nifedinpine as well as suitable substances and salts thereof mentioned in Pharm. Int., Nov. 85, pages 267-271, Barney H. Hunter and Robert L. Talbert, nitroglycerine, erythrityl tetranitrate, strychnine and salts thereof, lidocaine, tetracaine hydrochloride, etorphine hydrochloride, atropine, insulin, enzymes (for instance papain, trypsin, amyloglucosidase, glucoseoxidase, streptokinase, streptodornase, dextranase, alpha amylase), polypeptides (oxytocin, gonadorelin, (LH.RH), desmopressin acetate (DDAVP), isoxsuprine hydrochloride, ergotamine compounds, chloroquine (phosphate, sulfate), isosorbide, demoxytocin, heparin.

Other active ingredients include beta-lupeol, Letigen®, Sildenafil citrate and derivatives thereof.

Further examples of active ingredients include vitamins. Vitamins include A, B1, B2, B6, B12, Folinic acid, Folic acid, niacin, Pantothenic acid, biotine, C, D, E, K. Minerals include Calcium, phosphor, magnesium, iron, Zinc, Copper, Iod, Mangan, Crom, Selene, Molybden. Other active ingredients include: Q10®, enzymes. Natural drugs including Ginkgo biloba, ginger, and fish oil.

Further examples of active ingredients include migraine drugs such as Serotonin antagonists: Sumatriptan, Zolmitriptan, Naratriptan, Rizatriptan, Eletriptan; nausea drugs such as Cyclizin, Cinnarizin, Dimenhydramin, Difenhydrinat; hay fever drugs such as Cetrizin, Loratidin, pain relief drugs such as Buprenorfin, Tramadol, oral disease drugs such as Miconazol, Amphotericin B, Triamcinolonaceton; and the drugs Cisaprid, Domperidon, Metoclopramid. In a preferred embodiment the invention relates to the release of Nicotine and its salts.

In an advantageous embodiment of the invention the active ingredient is selected from active ingredients for the throat selected from acetylcysteine, ambroxol, amylmetacresol, benzocaine, bisacodyl, bismuth subsalicylate, bromhexine, cetirizine, dextromethorphan hydrobromide, 2,4-dichlorobenzyl alcohol, doxylamine succinate, eucalyptus oil, flurbiprofen, glycerin, hexylresorcinol, lidocaine, menthol, myrrh, paracetamol, pectin, peppermint oil, phenol, phenylephrine, povidone-iodine, pseudoephedrine, ranitidine, simethicone, sodium docusate, spearmint, zinc, or any combination thereof; active ingredients for the gastrointestinal tract selected from alginate, atenolol, aspirin (acetylsalicylic acid), ampicillin, aminosalicylates, anhydrous citric acid, aspirin, bisacodyl, bismuth subsalicylate, bupropion, caffeine, calcium, calcium carbonate, cetirizine, cimetidine, cisapride, clarithromycin, desloratadine, dexlansoprazole, diphenhydramine HCl, diphenhydramine citrate, dimenhydrinate, docusate erythromycin, dopamine, esomeprazole, famotidine, fexofenadine HCl, guaifenesin, hydrotalcite, ibuprofen, ketoprofen, lactase enzyme, lansoprazole, loratadine, lorcaserin, loperamide, loperamide HCl, magnesium, magnesium carbonate, magnesium hydroxide, melatonin, methamphetamine HCl, metoclopramide, metronidazole, montelukast, mycostatin, naltrexone, naproxen, naproxen sodium, nizatidine, omeprazole, ondansetron, orlistat, pantoprazole, paracetamol (acetaminophen), pectin, phentermine HCl, polypodium leucotomos, prednisolone, prednisone, progesterone, propranolol, propantheline bromide, pseudoephedrine HCl, phentermine, rabeprazole, ranitidine, roflumilast, scopoloamine butyl hydroxide, simethicone, sodium, sodium bicarbonate, sodium docusate, sumatriptan, testosterone, tetracycline, topiramate, vitamin A, vitamin B, vitamin B12, vitamin C (ascorbic acid), vitamin D, and vitamin E, vitamin K, or any combination thereof, and active ingredients for buccal absorption selected from atenolol, baclofen, caffeine, carvedilol, chlorpheniramine, chlorpheniramine maleate, fluticasone propionate, maleate, desmopressin, diltiazem hydrochloride, doxylamine succinate, mycostatin, nicotine, nifedipine, nitroglycerin, omeprazole, ondansetron, oxymetazoline HCl, oxytocin, phenylephrine, piroxicam, prednisone, propranolol, salbutamol sulphate, scopoloamine butyl hydroxide, sumatriptan, triamcinolonacetonid, and any combination thereof.

In one aspect of the invention, the “tablet” is intended to mean a “fast disintegrating tablet” (“FDT”), or similar wording, such as “orally disintegrating tablet” (“ODT”). If not stated otherwise, if the tablet according to the invention is made as one module, contrary to two or more modules, then the tablet is intended to be an FDT tablet. If on the other hand, the tablet is made of more than one module, such as two modules, such additional module is intended to be a “lozenge” module or “chewing gum module”, which provides a longer disintegration time compared to the FDT module according to the invention. The combination of an “FDT” module and a “lozenge” module (or a “chewing gum module) contributes to another aspect of the invention. A “lozenge” module or “chewing gum module” according to the invention may also comprise elements from the “FDT” modules but is generally different in composition, providing an extended disintegration time.

The term “module” is generally intended to be composed of a composition of matter with substantially the same characteristics throughout the module. Hence, if two module are present, then the two modules are different in composition and generally have two different characteristics throughout each module. In the present context, if only one module is present, then this module is considered an FDT tablet. On the other hand, if two modules are present, then the tablet is composed of an FDT tablet or FDT tablet module fused with a lozenge tablet or lozenge module. The term “fused” is intended to mean that the tablet is gathered together by means of compression force. Usually, if two modules are present, the lozenge module is made as the first module and the FDT module is made as the second module. The tablet may be composed of more than two module. The lozenge module may in certain embodiments be replaced by a gum base module. In the present context, the invention provides an attractive bi-phasic delivery of masking, even if the delivery of nicotine is “single-phased”.

### EXAMPLES

### Example 1

### Preparation of Dextrose Tablets

In a first step, dextrose was added to a mixing container. Active ingredients, flavors, high-intensity sweeteners, and optional other components were added to the container. The mixture was sieved and tumbled in a FUCHS Mixomat-A at approximately 25 rpm for 4 minutes. A processing aid was added and the mixture was tumbled at approximately 25 rpm for another 1 minute. Hereafter, the mixture was ready for tableting.

The mixture was subsequently led to a standard tablet pressing machine (3090i, available from Fette GmbH) comprising dosing apparatus (P 3200 C, available from Fette GmbH, Germany) and pressed into two-layered tablets. Alternatively, a Riva Picoola Bi-layer DC-PL-015 was used. The tablets were pressed using a pre-compression force of about 2.2 kN to form a first layer. After pre-compression of the first layer another portion of powder was added in the apparatus and a compression force of 10-30 kN was applied, unless otherwise indicated. There were 11 punches on the rotor, and the rotor speed used was 5 rpm. The individual tablets had a weight of approx. 1500 mg where the layers constituted weight ratios of about 1:1, unless otherwise stated in the examples below. Punch used: 15.00 mm, circular, shallow concave, B tooling.

Dextrose applied according to the examples was C*dex™ 02001 commercially available from Cargill, or C*dex™ 02030 provided by Cargill, or C*dex™ 02032 provided by Cargill, or Cerelose® dextrose 020010 provided by Ingredion, or Royal-TR provided by Ingredion. In some examples, separate binders applied were applied, such as HPC and HPMC. HPC was available as Klucel Nutra D from Ashland. HPMC was available as Methocel 4 KM from Dow. When microcrystalline cellulose was applied as a comparative binder, it was Avicel PH-102 commercially available from Dupont. When maltodextrin was applied as a binder, it was C*dry™ MD from Cargill.

### Example 2

### Composition of Two-Layered Dextrose Tablets in Presence of an Active Ingredient in Different Amounts

Dextrose tablets based on the procedure in Example 1 were made with the formulations outlined in the examples below. In all of the tablet examples, the amount of the various ingredients is given as % by weight of each layer of the tablet.

Unless specifically indicated, the same components were applied in the different layers of the tablet except for the flavor type which may be varied in different layers. Color in minute amounts was optionally added to one or more layers. The layers had the same weight percentages by weight of the tablet.

TABLE 1

| | Tablet Number | | | | |
|---|---|---|---|---|---|
| | 100 | 101 | 102 | 103 | 104 |
| | Content | Content | Content | Content | Content |
| Raw material name | [%] | [%] | [%] | [%] | [%] |
| Dextrose* | 94.9 | 90 | 75 | 65 | 55 |
| Active ingredient** | 0.1 | 5 | 20 | 30 | 40 |
| Flavors/high-intensity | 4.0 | 4.0 | 4.0 | 4.0 | 4.0 |
| sweeteners | | | | | |
| Processing aids | 1.0 | 1.0 | 1.0 | 1.0 | 1.0 |
| Total | 100 | 100 | 100 | 100 | 100 |

Dextrose was C*dex ™ 02032 commercially available from Cargill.
**The active ingredient was vitamin C available from DSM. The first layer of the tablets was pre-compressed with a force of about 2.2 kN and the second layer was compressed with a force of about 20 kN.

Tablets 100-104 were repeated as tablets 100A-104A using C*dex™ 02030 available from Cargill and as tablets 100B-104B using Royal-T® available from Ingredion.

### Example 3

Composition of Two-Layered Dextrose Tablets with Different Grades of Dextrose in Each Layer

Dextrose tablets based on the procedure in Example 1 were made with the formulations outlined in the examples below. In all of the tablet examples, the amount of the various ingredients is given as % by weight of each layer of the tablet.

Unless specifically indicated, the same components were applied in the different layers of the tablet except for the flavor type which may be varied in different layers. Color in minute amounts was optionally added to one or more layers. The layers had the same weight percentages by weight of the tablet.

TABLE 2

| | Tablet Number | | | | |
|---|---|---|---|---|---|
| | 110 | 111 | 112 | 113 | 114 |
| | Content | Content | Content | Content | Content |
| Raw material name | [%] | [%] | [%] | [%] | [%] |
| Dextrose* | 94.9 | 90 | 75 | 65 | 55 |
| Active ingredient** | 0.1 | 5 | 20 | 30 | 40 |
| Flavors/high-intensity | 4.0 | 4.0 | 4.0 | 4.0 | 4.0 |
| sweeteners | | | | | |
| Processing aids | 1.0 | 1.0 | 1.0 | 1.0 | 1.0 |
| Total | 100 | 100 | 100 | 100 | 100 |

*Dextrose in layer 1 was C*dex ™ 02032 commercially available from Cargill and dextrose in layer 2 was Royal-T ® provided by Ingredion.
**The active ingredient was vitamin C available from DSM. The first layer of the tablets was pre-compressed with a force of about 2.2 kN and the second layer was compressed with a force of about 20 kN.

### Example 4

Composition of Two-Layered Dextrose Tablets with Different Grades of Dextrose in Each Layer

Dextrose tablets based on the procedure in Example 1 were made with the formulations outlined in the examples below. In all of the tablet examples, the amount of the various ingredients is given as % by weight of each layer of the tablet.

Unless specifically indicated, the same components were applied in the different layers of the tablet except for the flavor type which may be varied in different layers.

Color in minute amounts was optionally added to one or more layers. The layers had the same weight percentages by weight of the tablet.

TABLE 3

| | Tablet Number | | | | |
|---|---|---|---|---|---|
| | 120 | 121 | 122 | 123 | 124 |
| | Content | Content | Content | Content | Content |
| Raw material name | [%] | [%] | [%] | [%] | [%] |
| Dextrose* | 94.9 | 90 | 75 | 65 | 55 |
| Active ingredient** | 0.1 | 5 | 20 | 30 | 40 |
| Flavors/high-intensity | 4.0 | 4.0 | 4.0 | 4.0 | 4.0 |
| sweeteners | | | | | |
| Processing aids | 1.0 | 1.0 | 1.0 | 1.0 | 1.0 |
| Total | 100 | 100 | 100 | 100 | 100 |

*Dextrose in layer 2 was C*dex ™ 02001 commercially available from Cargill and dextrose in layer 1 was Royal-T ® provided by Ingredion.
**The active ingredient was vitamin C available from DSM. The first layer of the tablets was pre-compressed with a force of about 2.2 kN and the second layer was compressed with a force of about 20 kN.

### Example 5

Composition of Two-Layered Dextrose Tablets with Different Grades of Dextrose in Each Layer

Dextrose tablets based on the procedure in Example 1 were made with the formulations outlined in the examples below. In all of the tablet examples, the amount of the various ingredients is given as % by weight of each layer of the tablet.

Unless specifically indicated, the same components were applied in the different layers of the tablet except for the flavor type which may be varied in different layers. Color in minute amounts was optionally added to one or more layers. The layers had the same weight percentages by weight of the tablet.

TABLE 4

| | Tablet Number | | | | |
|---|---|---|---|---|---|
| | 130 | 131 | 132 | 133 | 134 |
| | Content | Content | Content | Content | Content |
| Raw material name | [%] | [%] | [%] | [%] | [%] |
| Dextrose* | 94.9 | 93.9 | 89.9 | 84.9 | 79.9 |
| Active ingredient** | 0.1 | 0.1 | 0.1 | 0.1 | 0.1 |
| Binder*** (layer 1) | — | 1.0 | 5.0 | 10.0 | 15.0 |
| Flavors/high-intensity | 4.0 | 4.0 | 4.0 | 4.0 | 4.0 |
| sweeteners | | | | | |
| Processing aids | 1.0 | 1.0 | 1.0 | 1.0 | 1.0 |
| Total | 100 | 100 | 100 | 100 | 100 |

*Dextrose in layer 1 was C*dex ™ 02001 commercially available from Cargill and dextrose in layer 2 was Royal-T ® provided by Ingredion.
**The active ingredient was melatonin available from JiaHerb. The first layer of the tablets was pre-compressed with a force of about 2.2 kN and the second layer was compressed with a force of about 20 kN.
***The binder was HPC. No binder was added to layer 2, hence Royal-T ® was added in an amount of 95.9% in layer two of all tablets.

### Example 6

### Composition of Two-Layered Dextrose Tablets

Dextrose tablets based on the procedure in Example 1 were made with the formulations outlined in the examples below. In all of the tablet examples, the amount of the various ingredients is given as % by weight of each layer of the tablet.

Unless specifically indicated, the same components were applied in the different layers of the tablet except for the flavor type which may be varied in different layers. Color in minute amounts was optionally added to one or more layers. The layers had the same weight percentages by weight of the tablet.

TABLE 5

| | Tablet Number | | | | |
|---|---|---|---|---|---|
| | 140 | 141 | 142 | 143 | 144 |
| | Content | Content | Content | Content | Content |
| Raw material name | [%] | [%] | [%] | [%] | [%] |
| Dextrose* | 94.9 | 93.9 | 89.9 | 84.9 | 79.9 |
| Active ingredient** | 0.1 | 0.1 | 0.1 | 0.1 | 0.1 |
| Binder*** (layer 1 and 2) | — | 1.0 | 5.0 | 10.0 | 15.0 |
| Flavors/high-intensity | 4.0 | 4.0 | 4.0 | 4.0 | 4.0 |
| sweeteners | | | | | |
| Processing aids | 1.0 | 1.0 | 1.0 | 1.0 | 1.0 |
| Total | 100 | 100 | 100 | 100 | 100 |

*Dextrose in layer 1 and 2 was C*dex ™ 02001 commercially available from Cargill.
**The active ingredient was melatonin available from JiaHerb. The first layer of the tablets was pre-compressed with a force of about 2.2 kN and the second layer was compressed with a force of about 20 kN.

Tablets 140-144 were repeated as tablets 140A-144A using Cerelose® dextrose 020010 available from Ingredion.

### Example 7

Composition of two-layered dextrose tablets with different actives in each layer Dextrose tablets based on the procedure in Example 1 were made with the formulations outlined in the examples below. In all of the tablet examples, the amount of the various ingredients is given as % by weight of each layer of the tablet.

Unless specifically indicated, the same components were applied in the different layers of the tablet except for the flavor type which may be varied in different layers. Color in minute amounts was optionally added to one or more layers. The layers had the same weight percentages by weight of the tablet.

TABLE 6

| | Tablet Number | | | | |
|---|---|---|---|---|---|
| | 150 | 151 | 152 | 153 | 154 |
| | Content | Content | Content | Content | Content |
| Raw material name | [%] | [%] | [%] | [%] | [%] |
| Dextrose* | 94.9 | 90 | 75 | 65 | 55 |
| Active ingredient** | 0.1 | 5 | 20 | 30 | 40 |
| Flavors/high-intensity | 4.0 | 4.0 | 4.0 | 4.0 | 4.0 |
| sweeteners | | | | | |
| Processing aids | 1.0 | 1.0 | 1.0 | 1.0 | 1.0 |
| Total | 100 | 100 | 100 | 100 | 100 |

*Dextrose was C*dex ™ 02032 commercially available from Cargill.
**The active ingredient in layer 1 was L-theanine available from Sichuan Tongsheng and the active ingredient in layer 2 was GABA available from Kaiyuan Dayou. The first layer of the tablets was pre-compressed with a force of about 2.2 kN and the second layer was compressed with a force of about 20 kN.

### Example 8

Composition of two-layered dextrose tablets with different actives in each layer Dextrose tablets based on the procedure in Example 1 were made with the formulations outlined in the examples below. In all of the tablet examples, the amount of the various ingredients is given as % by weight of each layer of the tablet. Unless specifically indicated, the same components were applied in the different layers of the tablet except for the flavor type which may be varied in different layers. Color in minute amounts was optionally added to one or more layers. The layers had the same weight percentages by weight of the tablet.

TABLE 7

| | Tablet Number | | | | |
|---|---|---|---|---|---|
| | 160 | 161 | 162 | 163 | 164 |
| | Content | Content | Content | Content | Content |
| Raw material name | [%] | [%] | [%] | [%] | [%] |
| Dextrose* | 94.9 | 90 | 75 | 65 | 55 |
| Active ingredient** | 0.1 | 5 | 20 | 30 | 40 |
| Flavors/high-intensity | 4.0 | 4.0 | 4.0 | 4.0 | 4.0 |
| sweeteners | | | | | |
| Processing aids | 1.0 | 1.0 | 1.0 | 1.0 | 1.0 |
| Total | 100 | 100 | 100 | 100 | 100 |

*Dextrose was C*dex ™ 02032 commercially available from Cargill.
**The active ingredient in layer 1 was Vitamin C available from DSM and the active ingredient in layer 2 was calcium carbonate available from Nutrigranulation. The first layer of the tablets was pre-compressed with a force of about 2.2 kN and the second layer was compressed with a force of about 20 kN.

### Example 9

### Composition of Two-Layered Dextrose Tablets in Presence of an Active Ingredient in Different Amounts

Dextrose tablets based on the procedure in Example 1 were made with the formulations outlined in the examples below. In all of the tablet examples, the amount of the various ingredients is given as % by weight of each layer of the tablet. Unless specifically indicated, the same components were applied in the different layers of the tablet except for the flavor type which may be varied in different layers. Color in minute amounts was optionally added to one or more layers. The layers had the same weight percentages by weight of the tablet.

TABLE 8

| | Tablet Number | | | | |
|---|---|---|---|---|---|
| | 170 | 171 | 172 | 173 | 174 |
| | Content | Content | Content | Content | Content |
| Raw material name | [%] | [%] | [%] | [%] | [%] |
| Dextrose* | 94.9 | 90 | 75 | 65 | 55 |
| Active ingredient** | 0.1 | 5 | 20 | 30 | 40 |
| Flavors/high-intensity | 4.0 | 4.0 | 4.0 | 4.0 | 4.0 |
| sweeteners | | | | | |
| Processing aids | 1.0 | 1.0 | 1.0 | 1.0 | 1.0 |
| Total | 100 | 100 | 100 | 100 | 100 |

Dextrose was C*dex ™ 02032 commercially available from Cargill.
**The active ingredient was calcium carbonate available from Nutrigranulation. The first layer of the tablets was pre-compressed with a force of about 2.2 kN and the second layer was compressed with a force of about 20 kN.

Tablets 170-174 were repeated as tablets 170A-174A using C*dex™ 02030 available from Cargill and as tablets 170B-174B using Royal-T® available from Ingredion.

### Example 10

### Evaluations of Tablets

For each version of the tablets, a breaking point test, a friability test and a dissolution time measurement were performed. For measuring breaking point, a PTB 311 from Pharma Test was used.

The friability test was made in accordance to European Pharmacopoeia 9.1, test method 2.9.7. by using a pharmaceutical friability-tester PTF 10E from Pharma Test.

To test dissolution time, the following method was used. 15 mL of 0.02 M potassium dihydrogen phosphate-buffer (pH adjusted to 7.4) is added to 50 mL of water in a measuring tube with a screw cap. The tablet is inserted in the measuring tube and the screw cap is fastened. The measuring tube is fixated horizontally. The measuring tube is vibrated at about 110 RPM such that the tablet can move back and forth in the measuring tube. The measuring tube is vibrated until the tablet or module thereof in question is completely dissolved and the time of vibration is noted as the dissolution time.

### Example 11

### Sensorial Evaluation of Tablets

Sensoric tests were performed to reveal very important characteristics and properties of the tablets. These sensoric parameters are important as indicators of the structure of the tablet composition. The test set-up was composed of 8 test persons in a test panel. All of the test persons were healthy individuals appointed on an objective basis according to specified requirements. The sensory analysis was performed according to ISO 4121-2003 in testing conditions following ISO 8589. The result is an average of the results of the 8 individuals.

The test persons gave a rating from “+” to “+++++”, where “+” is poor and “+++++” is excellent. “0” indicated that it was not tested.

Six different parameters were tested in a test panel:
“Ease of chewing into liquid”—the impression of the tablet when placed in the mouth and chewed with respect to easiness of chewing the product into liquid. The criteria is that upon completion, there is no sense of particles in the mouth, and the powder of the tablet has dissolved into liquid.
“Liquid feeling”—the impression of the tablet when placed in the mouth and chewed with respect to the sense of liquid in the mouth. For instance, if more liquid is sensed during and/or after chewing, then the score is high.
“Mouthfeel”—the overall impression of the tablet during chewing with respect to mouthfeel, including melting and tacking sensations. A high scoring mouthfeel is associated with a clean liquid (no sense of particles), no tablet residuals sticking in teeth and a creamy feeling (higher viscosity than water). On the contrary, a low scoring mouthfeel is associated with sense of particles in the liquid (incomplete dissolution), tablet residuals sticking in the teeth and a watery feeling of the liquid.
“Overall taste”—the overall impression of the taste of the tablet during chewing. For instance, if the taste was decreasing rapidly, a very low rating was given.
“Overall sweetness”—the overall impression of the sweetness of the tablet during chewing. For instance, if the sweetness was decreasing rapidly, a very low rating was given.
“Overall sourness”—the overall impression of the sourness of the tablet during chewing. For instance, if the sourness was decreasing rapidly, a very low rating was given.

### Example 12

### Results of Two-Layered Dextrose Tablets in Presence of an Active Ingredient in Different Amounts

TABLE 9

| Tablet Number | 100 | 101 | 102 | 103 | 104 |
|---|---|---|---|---|---|
| Ease of chewing | +++++ | +++++ | +++++ | ++++ | +++ |
| into liquid | | | | | |
| Mouthfeel | +++++ | +++++ | +++++ | ++++ | ++++ |
| Comments | Easy, no | Easy, no | Easy, no | Easy, no | Easy, no |
| | sticking, no | sticking, no | sticking, no | sticking, no | sticking, no |
| | grease or | grittiness | grittiness | grittiness | grittiness |
| | grittiness | | | | |
| Friability | <1 | <1 | <1 | <1 | <1 |

Tested in compliance with Examples 10 and 11.

Generally, the results reveal that the content of actives was suitable in the range from 0.1 to 40% by weight of the active ingredient when compressed in a two-layered tablet according to the invention. It was a surprise that a high content of active ingredients was suitable in the two-layered configuration.

### Example 13

Results of Two-Layered Dextrose Tablets with Different Grades of Dextrose in Each Layer

TABLE 10

| Tablet Number | 110 | 111 | 112 | 113 | 114 |
|---|---|---|---|---|---|
| Ease of chewing | +++++ | +++++ | +++++ | +++++ | ++++ |
| into liquid | | | | | |
| Mouthfeel | +++++ | +++++ | ++++ | ++++ | +++ |
| Comments | Easy, no | Easy, no | Easy, no | Easy, no | Easy, no |
| | sticking, no | sticking, no | sticking, no | sticking, no | sticking, no |
| | grease or | grittiness | grittiness | grittiness | grittiness |
| | grittiness | | | | |
| Friability | <1 | <1 | <1 | <1 | <1 |

Tested in compliance with Examples 10 and 11.

Generally, the results reveal that the content of actives was suitable in the range from 0.1 to 40% by weight of the active ingredient when compressed in a two-layered tablet according to the invention. It was a surprise that a high content of active ingredients was suitable in the two-layered configuration.

### Example 14

Results of Two-Layered Dextrose Tablets with Different Grades of Dextrose in Each Layer

TABLE 11
Tested in compliance with Examples 10 and 11.

| Tablet Number | 120 | 121 | 122 | 123 | 124 |
|---|---|---|---|---|---|
| Ease of chewing | +++++ | +++++ | +++++ | ++++ | ++++ |
| into liquid | | | | | |
| Mouthfeel | +++++ | +++++ | ++++ | +++++ | ++++ |
| Comments | Easy, no | Easy, no | Easy, no | Easy, no | Easy, no |
| | sticking, no | sticking, no | sticking, no | sticking, no | sticking, no |
| | grease or | grittiness | grittiness | grittiness | grittiness |
| | grittiness | | | | |
| Friability | <1 | <1 | <1 | <1 | <1 |

Generally, the results reveal that the content of actives was suitable in the range from 0.1 to 40% by weight of the active ingredient when compressed in a two-layered tablet according to the invention. Additionally, the results reveal that the two different grades in the different layers of the tablets were suitable according to the invention.

### Example 15

Results of two-layered dextrose tablets with different grades of dextrose in each layer

TABLE 12
Tested in compliance with Examples 10 and 11.

| Tablet Number | 130 | 131 | 132 | 133 | 134 |
|---|---|---|---|---|---|
| Ease of chewing | +++++ | +++++ | +++++ | +++++ | ++++ |
| into liquid | | | | | |
| Mouthfeel | +++++ | +++++ | +++++ | ++++ | ++++ |
| Comments | Easy, no | Easy, no | Easy, no | Easy, no | Easy, no |
| | sticking, no | sticking, no | sticking, no | sticking, no | sticking, no |
| | grease or | grittiness | grittiness | grittiness | grittiness |
| | grittiness | | | | |
| Friability | <1 | <1 | <1 | <1 | <1 |

Generally, the results reveal that adding a separate binder in layer 1 was suitable to obtain a good result when compressed in a two-layered tablet according to the invention.

### Example 16

Results of Two-Layered Dextrose Tablets with Different Grades of Dextrose in Each Layer

TABLE 13
Tested in compliance with Examples 10 and 11.

| Tablet Number | 140 | 141 | 142 | 143 | 144 |
|---|---|---|---|---|---|
| Ease of chewing | +++++ | +++++ | +++++ | +++++ | ++++ |
| into liquid | | | | | |
| Mouthfeel | +++++ | ++++ | +++++ | ++++ | ++++ |
| Comments | Easy, no | Easy, no | Easy, no | Easy, no | Easy, no |
| | sticking, no | sticking, no | sticking, no | sticking, no | sticking, no |
| | grease or | grittiness | grittiness | grittiness | grittiness |
| | grittiness | | | | |
| Friability | <1 | <1 | <1 | <1 | <1 |

Generally, the results reveal that adding a separate binder in both layer 1 and 2 was suitable to obtain a good result when compressed in a two-layered tablet according to the invention.

### Example 17

Results of Two-Layered Dextrose Tablets with Different Actives in Each Layer

TABLE 14
Tested in compliance with Examples 10 and 11.

| Tablet Number | 150 | 151 | 152 | 153 | 154 |
|---|---|---|---|---|---|
| Ease of chewing | +++++ | +++++ | +++++ | +++++ | ++++ |
| into liquid | | | | | |
| Mouthfeel | +++++ | +++++ | +++++ | +++++ | ++++ |
| Comments | Easy, no | Easy, no | Easy, no | Easy, no | Easy, no |
| | sticking, no | sticking, no | sticking, no | sticking, no | sticking, no |
| | grease or | grittiness | grittiness | grittiness | grittiness |
| | grittiness | | | | |
| Friability | <1 | <1 | <1 | <1 | <1 |

Generally, the results reveal that the content of actives was suitable in the range from 0.1 to 40% by weight of the active ingredient when compressed in a two-layered tablet according to the invention.

### Example 18

Results of Two-Layered Dextrose Tablets with Different Actives in Each Layer

TABLE 15
Tested in compliance with Examples 10 and 11.

| Tablet Number | 160 | 161 | 162 | 163 | 164 |
|---|---|---|---|---|---|
| Ease of chewing | +++++ | +++++ | +++++ | +++++ | ++++ |
| into liquid | | | | | |
| Mouthfeel | +++++ | +++++ | +++++ | ++++ | ++++ |
| Comments | Easy, no | Easy, no | Easy, no | Easy, no | Easy, no |
| | sticking, no | sticking, no | sticking, no | sticking, no | sticking, no |
| | grease or | grittiness | grittiness | grittiness | grittiness |
| | grittiness | | | | |
| Friability | <1 | <1 | <1 | <1 | <1 |

Generally, the results reveal that the content of actives was suitable in the range from 0.1 to 40% by weight of the active ingredient when compressed in a two-layered tablet according to the invention.

### Example 19

Results of Two-Layered Dextrose Tablets with Presence of an Active Ingredient in Different Amounts

TABLE 16
Tested in compliance with Examples 10 and 11.

| Tablet Number | 170 | 171 | 172 | 173 | 174 |
|---|---|---|---|---|---|
| Ease of chewing | +++++ | +++++ | +++++ | +++++ | ++++ |
| into liquid | | | | | |
| Mouthfeel | +++++ | +++++ | +++++ | ++++ | ++++ |
| Comments | Easy, no | Easy, no | Easy, no | Easy, no | Easy, no |
| | sticking, no | sticking, no | sticking, no | sticking, no | sticking, no |
| | grease or | grittiness | grittiness | grittiness | grittiness |
| | grittiness | | | | |
| Friability | <1 | <1 | <1 | <1 | <1 |

Generally, the results reveal that the content of actives was suitable in the range from 0.1 to 40% by weight of the active ingredient when compressed in a two-layered tablet according to the invention.

### Example 20

Composition of Dextrose Tablets with Different Active Ingredients Focused on Energy

Dextrose tablets based on the procedure in Example 1 were made with the formulations outlined in the examples below, here including caffeine in an amount of 100 mg and optionally vitamin B premix in an amount of 15 mg as active ingredient. In all of the tablet examples, the amount of the various ingredients is given as % by weight of each layer of the tablet.

Unless specifically indicated, the same components were applied in the different layers of the tablet except for the flavor type which may be varied in different layers. Color in minute amounts was optionally added to one or more layers. The layers had the same weight percentages by weight of the tablet.

TABLE 17

| | Tablet Number | | | |
|---|---|---|---|---|
| | 180 | 181 | 182 | 183 |
| | Content | Content | Content | Content |
| Raw material name | [%] | [%] | [%] | [%] |
| Dextrose | 90.8* | 89.8* | 90.8** | 89.8** |
| Caffeine*** | 6.8 | 6.8 | 6.8 | 6.8 |
| Vitamin B**** | — | 1.0 | — | 1.0 |
| Flavors/high-intensity sweeteners | 1.4 | 1.4 | 1.4 | 1.4 |
| Processing aids | 1.0 | 1.0 | 1.0 | 1.0 |
| Total | 100 | 100 | 100 | 100 |

*Dextrose was C*dex ™ 02032 commercially available from Cargill.
**Dextrose was Royal-T ® provided by Ingredion.
***Caffeine available from Siegfried,
****Vitamin B premix available from DSM. The first layer of the tablets was pre-compressed with a force of about 2.2 kN and the second layer was compressed with a force of about 20 kN.

### Example 21

Composition of Dextrose Tablets with Different Active Ingredients Focused on Energy

Dextrose tablets based on the procedure in Example 1 were made with the formulations outlined in the examples below, here including ginseng in an amount of 10 mg and optionally vitamin B premix in an amount of 10 mg as active ingredient. In all of the tablet examples, the amount of the various ingredients is given as % by weight of each layer of the tablet. For all samples in this example, the total tablet weight is 1000 mg.

Unless specifically indicated, the same components were applied in the different layers of the tablet except for the flavor type which may be varied in different layers.

Color in minute amounts was optionally added to one or more layers. The layers had the same weight percentages by weight of the tablet.

TABLE 18

| | Tablet Number | | | |
|---|---|---|---|---|
| | 190 | 191 | 192 | 193 |
| | Content | Content | Content | Content |
| Raw material name | [%] | [%] | [%] | [%] |
| Dextrose | 95.0* | 94.0* | 95.0** | 94.0** |
| Ginseng*** | 1.0 | 1.0 | 1.0 | 1.0 |
| Vitamin B**** | — | 1.0 | — | 1.0 |
| Flavors/high-intensity sweeteners | 3.0 | 3.0 | 3.0 | 3.0 |
| Processing aids | 1.0 | 1.0 | 1.0 | 1.0 |
| Total | 100 | 100 | 100 | 100 |

*Dextrose was C*dex ™ 02032 commercially available from Cargill.
**Dextrose was Royal-T ® provided by Ingredion.
***Ginseng available from Naturex,
****Vitamin B premix available from DSM. The first layer of the tablets was pre-compressed with a force of about 2.2 kN and the second layer was compressed with a force of about 20 kN.

### Example 22

Composition of Dextrose Tablets with Different Active Ingredients Focused on Energy

Dextrose tablets based on the procedure in Example 1 were made with the formulations outlined in the examples below, here including L-theanine in an amount of 100 mg and optionally caffeine in an amount of 50 mg as active ingredient. In all of the tablet examples, the amount of the various ingredients is given as % by weight of each layer of the tablet.

Unless specifically indicated, the same components were applied in the different layers of the tablet except for the flavor type which may be varied in different layers. Color in minute amounts was optionally added to one or more layers. The layers had the same weight percentages by weight of the tablet.

TABLE 19

| | Tablet Number | | | |
|---|---|---|---|---|
| | 200 | 201 | 202 | 203 |
| | Content | Content | Content | Content |
| Raw material name | [%] | [%] | [%] | [%] |
| Dextrose | 90.8* | 87.4* | 90.8** | 87.4** |
| L-theanine*** | 6.8 | 6.8 | 6.8 | 6.8 |
| Caffeine**** | — | 3.4 | — | 3.4 |
| Flavors/high-intensity sweeteners | 1.4 | 1.4 | 1.4 | 1.4 |
| Processing aids | 1.0 | 1.0 | 1.0 | 1.0 |
| Total | 100 | 100 | 100 | 100 |

*Dextrose was C*dex ™ 02032 commercially available from Cargill.
**Dextrose was Royal-T ® provided by Ingredion.
***L-theanine available from Sichuan Tongsheng,
****Caffeine available from Siegfried. The first layer of the tablets was pre-compressed with a force of about 2.2 kN and the second layer was compressed with a force of about 20 kN.

### Example 23

Composition of Dextrose Tablets with Different Active Ingredients Focused on Immune Stimulants

Dextrose tablets based on the procedure in Example 1 were made with the formulations outlined in the examples below, here including vitamin C in an amount of 500 mg or a herbal blend with vitamin C and other vitamins/minerals in an amount of 450 mg as active ingredient. In all of the tablet examples, the amount of the various ingredients is given as % by weight of each layer of the tablet.

Unless specifically indicated, the same components were applied in the different layers of the tablet except for the flavor type which may be varied in different layers. Color in minute amounts was optionally added to one or more layers. The layers had the same weight percentages by weight of the tablet.

TABLE 20

| | Tablet Number | | | |
|---|---|---|---|---|
| | 210 | 211 | 212 | 213 |
| | Content | Content | Content | Content |
| Raw material name | [%] | [%] | [%] | [%] |
| Dextrose | 63.8* | 67.1* | 63.8** | 67.1** |
| Vitamin C*** | 33.3 | — | 33.3 | — |
| Herbal blend with vitamins**** | — | 30.0 | — | 30.0 |
| Flavors/high-intensity sweeteners | 1.9 | 1.9 | 1.9 | 1.9 |
| Processing aids | 1.0 | 1.0 | 1.0 | 1.0 |
| Total | 100 | 100 | 100 | 100 |

*Dextrose was C*dex 02032 ™ commercially available from Cargill.
**Dextrose was Royal-T ® provided by Ingredion.
***Vitamin C available from DSM,
****Herbal blend with vitamins available from DSM. The first layer of the tablets was pre-compressed with a force of about 2.2 kN and the second layer was compressed with a force of about 20 kN.

### Example 24

Composition of Dextrose Tablets with Different Active Ingredients Focused on Cough and Cold Formulations

Dextrose tablets based on the procedure in Example 1 were made with the formulations outlined in the examples below, here including active ingredients suitable for treating cough and cold symptoms. In all of the tablet examples, the amount of the various ingredients is given as % by weight of each layer of the tablet.

Unless specifically indicated, the same components were applied in the different layers of the tablet except for the flavor type which may be varied in different layers. Color in minute amounts was optionally added to one or more layers. The layers had the same weight percentages by weight of the tablet. For all samples in this example, the total tablet weight is 1750 mg.

TABLE 21
It was secured that the binders were thoroughly mixed into the dry mixture.

| | Tablet Number | | | | | |
|---|---|---|---|---|---|---|
| | 220 | 221 | 222 | 223 | 224 | 225 |
| | Content | Content | Content | Content | Content | Content |
| Raw material name | [mg] | [mg] | [mg] | [mg] | [mg] | [mg] |
| Dextrose* | 662.5 | 655 | 500 | — | — | — |
| Dextrose** | — | — | — | 662.5 | 655 | 500 |
| HPC binder | 17.5 | 17.5 | 17.5 | 17.5 | 17.5 | 17.5 |
| Acetominophen*** | 125 | 125 | 125 | 125 | 125 | 125 |
| Dextromethorphan**** | — | 5 | 5 | — | 5 | 5 |
| Phenylephrine***** | — | 2.5 | 2.5 | — | 2.5 | 2.5 |
| Mannitol (Pearlitol DC300) | — | — | 155 | — | — | 155 |
| Flavors/high-intensity | 61.5 | 61.5 | 61.5 | 61.5 | 61.5 | 61.5 |
| sweeteners/fruit acids | | | | | | |
| Processing aids | 8.5 | 8.5 | 8.5 | 8.5 | 8.5 | 8.5 |
| Total layer weight | 875 | 875 | 875 | 875 | 875 | 875 |

*Dextrose was C*dex ™ 02001 commercially available from Cargill.
**Dextrose was Emdex commercially available from JRS Pharma.
***Acetominophen available from Mallinckrodt,
****Dextromethorphan HBr available from LGM Pharma,
*****Phenylephrine HCl available from Siegfried.
The first layer of the tablets was pre-compressed with a force of about 2.2 kN and the second layer was compressed with a force of about 30 kN.

### Example 25

Results of Comparison of Dextrose Tablet 211 with a Commercially Available Product Containing Immune Stimulants

TABLE 22
Tested in compliance with Examples 10 and 11.

| Tablet Number | 211 | Airborne |
|---|---|---|
| Ease of chewing | +++++ | +++ |
| into liquid | | |
| Mouthfeel | +++++ | ++ |
| Comments | Good | Crumble |
| | mouthfeel, | mouthfeel, |
| | almost no | sandy |
| | sandy | mouthfeel |
| | mouth-feel | |

Generally, the results reveal that dextrose tablet 211 was providing a superior mouthfeel and liquifying sensation compared to the commercially available product Airborne which contains the same type of actives as dextrose tablet 211.

### Example 26

Comparison of Two-Layered Tablets with One Layered Tablets

In the following example, the two-layered tablets according to the invention were compared to one-layered compressed tablets that were made by the same tools and conditions as outlined in Example 1. All tablets were prepared and made according to Example 1 but the one layered tablets were only made by adding one portion of tablet material and applying one varying main compression force to the tablets (without a pre-compression force), whereas the two-layered tablets were made by applying a pre-compression force of about 2.2 kN on a first portion of material to form a first layer for all tablets and a varying main compression force applied after adding a second portion of material to form the final two-layered tablets.

TABLE 23

| | Tablet Number | | | | | |
|---|---|---|---|---|---|---|
| Raw | 230* | 231* | 232* | 233** | 234** | 235** |
| material | Content | Content | Content | Content | Content | Content |
| name | [%] | [%] | [%] | [%] | [%] | [%] |
| C*dex ™ | 100 | — | — | 100 | — | — |
| 02001 | | | | | | |
| C*dex ™ | — | 100 | — | — | 100 | — |
| 02032 | | | | | | |
| Royal-T ® | — | — | 100 | — | — | 100 |
| Total | 100 | 100 | 100 | 100 | 100 | 100 |

*one-layered tablets.
**two-layered tablets.
Dextrose was C*dex ™ 02001 or C*dex ™ 02032 commercially available from Cargill or Royal-T ® commercially available from Ingredion.

### Example 27

### Friability of One-Layered Tablets of Example 26

TABLE 24

| Compression force (kN) | 10 | 20 | 30 |
|---|---|---|---|
| Friability of 230* | 6.7 | 1.6 | 0.5 |
| Friability of 231* | 3.8 | 0.5 | 0.5 |
| Friability of 232* | 3.0 | 0.5 | 0.1 |

*Dextrose was C*dex ™ 02001 or C*dex ™ 02032 commercially available from Cargill or Royal-T ® commercially available from Ingredion.

Generally, it was seen that the friability for one-layered tablets was on a lower level with respect to the dextrose grades C*dex 02032 and Royal-T compared to the grade C*dex 02001. Also, a low friability level was already obtained by a compression force of about 20 kN for all tablets.

### Example 28

### Tablet Hardness of One-Layered Tablets of Example 26

TABLE 25

| Compression force (kN) | 10 | 20 | 30 |
|---|---|---|---|
| Tablet hardness (N) of 230* | 34 | 82 | 124 |
| Tablet hardness (N) of 231* | 39 | 82 | 104 |
| Tablet hardness (N) of 232* | 47 | 119 | 204 |

*Dextrose was C*dex ™ 02001 or C*dex ™ 02032 commercially available from Cargill or Royal-T ® commercially available from Ingredion.

Generally, it was seen that the hardness for one-layered tablets was on a similar or higher level with respect to the dextrose grades C*dex 02032 and Royal-T compared to the grade C*dex 02001.

### Example 29

### Friability of Two-Layered Tablets of Example 26

TABLE 26

| Compression force (kN) | 10 | 20 | 30 |
|---|---|---|---|
| Friability of 233* | 5.7 | 1.7 | 21 |
| Friability of 234* | 3.0 | 0.7 | 0.1 |
| Friability of 235* | 3.4 | 0.2 | 0.3 |

*Dextrose was C*dex ™ 02001 or C*dex ™ 02032 commercially available from Cargill or Royal-T ® commercially available from Ingredion.

To the surprise, it was seen that the friability of the different grades was very low at a compression force of about 20 kN with sample 235 having a significantly lower friability than sample 234 and an even more significantly lower friability than sample 233. For the grade with fine particles (C*dex 02001), the tablets were subject to capping at a compression force of about 30 kN, hence a very high friability resulted. This was a huge surprise since the same capping issue was not seen for one-layered tablets. It is noted that no separate binders were present in the samples.

### Example 30

### Tablet Hardness of Two-Layered Tablets of Example 26

TABLE 27

| Compression force (kN) | 10 | 20 | 30 |
|---|---|---|---|
| Tablet hardness (N) of 233* | 48 | 103 | 84 |
| Tablet hardness (N) of 234* | 48 | 102 | 139 |
| Tablet hardness (N) of 235* | 54 | 112 | 192 |

*Dextrose was C*dex ™ 02001 or C*dex ™ 02032 commercially available from Cargill or Royal-T ® commercially available from Ingredion.

To the surprise, it was seen that the different grades were close to each other at a compression force of about 20 kN, despite the observed huge difference in friability of the same tablets. It was expected to observe a similar difference in hardness in view of the difference in friability at 20 kN. For the grade with fine particles (C*dex 02001), the tablets were subject to capping at a compression force of about 30 kN, hence a very low hardness resulted, which was a huge surprise. The capping issue was not seen for one-layered tablets. It is noted that no separate binders were present in the samples.

1. An oral chewable tablet, comprising:
dextrose in an amount from 50 to 95% by weight of the tablet; and
one or more active ingredients in an amount from 0.1 to 50% by weight of the tablet, wherein the oral chewable tablet comprises:
at least two compressed layers containing said dextrose and the one or more active ingredients, the first of the compressed layers being cohered to and adjacent to the second layer.

2. The oral chewable tablet according to claim 1, wherein the tablet consists of two layers.

3. The oral chewable tablet according to claim 1, wherein the weight ratio of the first layer relative to the second layer is from 1:10 to 10:1.

4. (canceled)

5. (canceled)

6. The oral chewable tablet according to claim 1, wherein the tablet consists of three layers where the first layer is the middle layer, and the second layer is cohered to and adjacent to one side of the first layer, and a third layer is cohered to and adjacent to the opposite side of the first layer.

7. The oral chewable tablet according to claim 6, wherein the weight ratio of the first layer relative to the second layer relative to the third layer is from 1:5:5 to 10:1:1.

8-10. (canceled)

11. The oral chewable tablet according to claim 1, wherein the first layer is pre-compressed by direct compression in a tableting machine and the second layer is compressed on top of the first layer in the tableting machine.

12-15. (canceled)

16. The oral chewable tablet according to claim 1, wherein the oral chewable tablet comprises one or more binders added separately in the formulation and being separate from any binder being integrated in other ingredients in the tablet.

17. The oral chewable tablet according to claim 1, wherein the oral chewable tablet comprises one or more binders in an amount of 2-15% by weight of the tablet added separately in the formulation and being separate from any binder being integrated in other ingredients in the tablet.

18-21. (canceled)

22. The oral chewable tablet according to claim 1, wherein the one or more binders comprises hydroxypropyl cellulose (HPC).

23. The oral chewable tablet according to claim 1, wherein the one or more binders comprises hydroxypropyl methylcellulose (HPMC).

24. The oral chewable tablet according to claim 1, wherein the one or more binders comprises maltodextrin.

25-32. (canceled)

33. The oral chewable tablet according to claim 1, wherein the oral chewable tablet is consisting essentially of dextrose, one or more active ingredients, one or more binders added separately in the formulation and being separate from any binder being integrated in other ingredients in the tablet, and auxiliary ingredients present up to about 5% by weight of the tablet.

34. The oral chewable tablet according to claim 1, wherein the oral chewable tablet is consisting essentially of dextrose, one or more active ingredients, and auxiliary ingredients present up to about 5% by weight of the tablet.

35-37. (canceled)

38. The oral chewable tablet according to claim 1, wherein the dextrose comprises dextrose selected from the group consisting of anhydrous dextrose, hydrated dextrose, dextrose monohydrate, and any combination thereof.

39. (canceled)

40. (canceled)

41. The oral chewable tablet according to claim 1, wherein the dextrose comprises at least 90% dextrose equivalents calculated on a dry basis.

42. (canceled)

43. The oral chewable tablet according to claim 1, wherein the dextrose comprises dextrose selected from the group consisting of oligomeric saccharides, microcrystalline dextrose, dextrate, and any combination thereof.

44. The oral chewable tablet according to claim 1, wherein the dextrose comprises 93 to 97% dextrose equivalents calculated on a dry basis.

45. (canceled)

46. (canceled)

47. The oral chewable tablet according to claim 1, wherein the dextrose comprises 100% dextrose equivalents calculated on a dry basis.

48-53. (canceled)

54. The oral chewable tablet according to claim 1, wherein the oral chewable tablet comprises different grades of dextrose in different layers of the tablet.

55-67. (canceled)

68. The oral chewable tablet according to claim 1, wherein the one or more active ingredients is present in an amount of 5 to 50% by weight of the tablet.

69-81. (canceled)

82. The oral chewable tablet according to claim 1, wherein the one or more active ingredients comprise an active ingredient selected from the group consisting of an immune supporting active ingredient, an energy stimulating active ingredient, a mixture of vitamins, minerals, and herbals, and any combination thereof.

83-89. (canceled)

90. The oral chewable tablet according to claim 1, wherein the oral chewable tablet comprises one or more disintegrants selected from the group consisting of sodium croscarmellose, crospovidone, sodium starch glycolate, and combinations thereof.

91-111. (canceled)
